(19) United States
(12) Patent Application Publication
(10) Pub. No.: US 2023/0119139 A1
(43) Pub. Date: Apr. 20, 2023

(54) SOFTWARE, HEALTH STATUS DETERMINATION DEVICE AND HEALTH STATUS DETERMINATION METHOD

(71) Applicant: Fuyo kaihatsu Co., Ltd., Fukuoka-shi (JP)

(72) Inventor: Shunsuke MAETA, Fukuoka-shi (JP)

(21) Appl. No.: 17/918,879

(22) PCT Filed: Aug. 26, 2020
(86) PCT No.: PCT/JP2020/032219

§ 371 Date: Oct. 13, 2022

(51) Int. Cl. A61B 5/00; A61B 5/0205; G16H 50/30; A61B 5/145

(52) U.S. Cl. CPC A61B 5/7275; A61B 5/02055; G16H 50/30; A61B 5/14542

(57) ABSTRACT

A health condition determination device analyzes short-term individual vital signs obtained by values of 4 times of measurements to determine an abnormality in the health condition. The health condition determination device includes a calculation unit which executes each information processing function. The software makes the calculation unit of a tablet terminal to function as an information input unit, an information recording unit, a reference calculation unit, and a determination processing unit. The processing functions of the respective means perform transmission of information, information recording, determination of abnormality in vital signs, setting abnormality determination reference regarding vital signs, notification of abnormality determination result regarding vital signs, scoring based on the contents of vital information, setting of scoring conditions, determination of abnormality in score value information, setting determination reference of abnormality regarding score value, notification of determination result regarding score values, and the like.

### TECHNICAL FIELD

The present disclosure relates to software, a health condition determination device, and a health condition determination method. Specifically, the present disclosure relates to software, a health condition determination device, and a health condition determination method which reflect vital signs or daily conditions in consideration of the individual differences of the subjects, enable the subjects to determine the intra-individual variation more quickly with high accuracy, and contribute to the subject's health care or the provision of medicine for the characteristics of each individual.

### BACKGROUND ART

Recently, the importance of “personalized medicine” in the medical field is increasing. The personalized medicine refers to “performing medical care suitable for the characteristics of each person”, called tailor-made medicine.

Until now, medical care has been performed based on consideration of mainly diseases, and the main purpose thereof is to search for the cause of the diseases or to develop a treatment method for the diseases. On the other hand, the status of a disease varies greatly from person to person, and it has been known that it is not always right to apply the same treatment even if the disease is the same.

However, in the conventional medical treatment, individual differences in therapeutic effects are considered unknown without observing; the treatment and the effects thereof, and thus, it is difficult to perform an optimal treatment plan for each individual.

Here, it is important to determine “biomarkers” different for each individual in realizing personalized medicine. In general, a biomarker is an indicator of a status of a particular disease or a status of life, and a research group at the National Institutes of Health defined biomarkers in 1988 as “characteristics that are objectively measured and evaluated as indicators of pharmacological responses to common biological processes, pathological processes, or therapeutic interventions.” Also, in the past, biomarkers mainly meant physiological indicators such as blood pressure, heart rate, and the like.

The reason why these vital signs can be used as biomarkers is because the vital signs such as body temperature, blood pressure, pulse, and respiratory rate have “intra-individual variation” that differ from person to person. In other words, the inventor thought that the method of change of vital signs differs depending on the subject, and the technique that contributed to the health management or diagnosis of the subject could be developed by appropriately determining and interpreting the method of change.

In addition, vital signs are widely used in daily health management in the fields, such as hospitals, nursing facilities, and home care, as an indicator that can easily determine the health conditions. However, essentially, when using the vital signs as biomarkers, it is necessary to perform analysis through the individual vital data so as to be tailor-made.

This is because each individual has individual differences, i.e., intra-individual variation unique to that person, and the elderly have characteristics different from those of general adults, such as a decrease in body temperature, increase in blood pressure, and decrease in pulse due to aging, and accordingly, there is a problem in determining the health conditions, based on absolute values.

This problem became known to many people when the Japanese Ministry of Health, Labor and Welfare withdrew the absolute value standard of 37.5° C. for fever due to the novel coronavirus and changed tailor-made's abnormality determination to “higher temperature than individual's normal temperature”.

(1) The average body temperature of Japanese is 36.9° C., and it changes by 1° C. within a day. (2) The distribution of body temperature varies from person to person, and there are many people whose average body temperature is 37.5° C. (3) The body temperature of the elderly decreases year by year due to the effects of aging. Through the above, there is a problem in uniformly determining the fever, based on the absolute value of 37.5° C.

However, in the case of “higher temperature than individual's normal temperature”, the numerical standard is not clear, so it was suggested through the Japan Medical Association COVID-19 Knowledgeable People's Meeting in July 2020, “2σ plus 0.5 degrees above the median normal distribution of temperature heat” should be regarded as fever (here, refers to heavy fever to watch out for), based on the papers (the “credit section of the average value ±2σ=95%” of each individual's body temperature is taken as the normal region) published in the Journal of the Chronic Medical Association of Japan by the two authors of the present disclosure.

The inventor not only verified the property that the vital signs of an individual's body temperature, blood pressure, pulse, and pulse pressure are always normally distributed except for special diseases, but also invented “vital sign abnormal value detection” technology that performs abnormality determination of the subject's vital signs (see, for example, patent document 1) and scoring determination (see, for example, patent document 2 and patent document 3), based on the ideas of including and distributing intra-individual variation unique to that person, by acquiring a certain number of data and preparing a determination reference (reference region) based on the average value (μ) and standard deviation (σ) of the data, and have been proved and published in a paper that the specificity of vital scoring for pneumonia hospitalization was 93% in the Welfare and Labor Science Study.

### PRIOR ART DOCUMENTS

### Patent Documents

Patent document 1: Specification of No. 6,350,959

Patent document 1: Specification of No. 6,512,648

Patent document 1: Specification of No. 6,551,959

### Non-Patent Documents

Non-patent document 1: “Early warning score”, [online], Wikipedia, the free encyclopedia, searched on Oct. 16, 2017, Internet URL:https://en.wikipedia.org/wiki/Early_warning_score

### DISCLOSURE OF INVENTION

### Technical Problem

The invention, “detection of abnormal values for vital signs”, has been utilized not only for applications that generate an alert for suspected corona by tailor-made's fever detection but also in electronic medical record cards, nursing soft bed sensors, and health homes, but has been considered to have a problem in that it takes 30 days or more through once-a-day measurement to establish a reference region (vital sign abnormality determination reference).

Therefore, taking into account the property that the vital signs of an individual's body temperature, blood pressure, pulse, and pulse pressure are always normally distributed except for special diseases, and furthermore, in order to enable rapid determination with a small number of data or a short period of time, examination for reducing measure points while maintaining accuracy was performed.

Here, the inventor analyzed vital data with little variance affecting 156 people for the value of vital signs measured once a day, and performed a statistical hypothesis test between the data group for 4 days and the data group for 30 days. As a result, there was no significant difference (P>0.05) even when compared with the data group for 30 days. In addition, verification was performed to prove that there was no difference (P is close to 1) in detection accuracy between the data group for 4 days and the data group for 30 days. Furthermore, details will be described later.

This proves that data for 4 days is sufficient to establish the reference region (determination reference) necessary to perform “vital sign abnormal value detection”. This makes it possible to detect vital sign abnormal values in tailor-made by using short-term vital data. In addition, since the data used for distribution is accumulated even after 4 days' worth of data, the technical result becomes the same as the previous technical result using data for 30 days or more when the data for 30 days (30 times) is accumulated.

This “vital sign abnormal value detection” technology can also be used for the “vital scoring” technique which assigns the abnormal values etc. which have been obtained thereby to a score distribution table and calculates a medical risk from the total score.

Also, in the medical field, a scoring method called an early warning score (hereinafter referred to as “EWS”) is used as a technique for rapidly evaluating the degree of a subject's illness.

The EWS is a technique for calculating a score according to a measurement result or an evaluation result of vital signs, based on the measurement of the subject's six major signs: respiratory rate (rpm), oxygen saturation (SpO2) (%), body temperature (° C.), blood pressure (mmHg), heart rate (bpm), and consciousness level (AVPU response: A: alert (normal), V: voice (responsive to voice), P: pain (responsive to pain), U: unresponsive), and determining the degree of disease by the total score of the score (refer, e.g., non-patent literature 1).

This EWS is based on the principle that the clinical degradation can be seen via changes in multiple physiological measurements and large changes within a single variable. In addition, when calculating a score from the measurement values of each vital sign, numerical values determined based on the result of the measurement value of a group (a plurality of subjects) is adopted.

For example, as shown in Table 1, in the case of body temperature, “36.0˜37.9° C.” is configured as a center range of the measurement value, and it becomes a score of zero (0) point in this range. In addition, for the upper and lower values, the measurement values of body temperature and the scores of the scoring are configured so that if it is “35.0˜35.9° C.” or “38.0˜38.9° C.”, the score becomes 1 point, if it is “34.0˜34.9° C.” or “a value exceeding 38.9° C.”, the score becomes 2 points, and if it is “less than 34° C.”, the score becomes 3 points. In addition, other vital signs are configured as shown in Table 1.

TABLE 1

| Score | 3 | 2 | 1 | 0 | 1 | 2 | 3 |
|---|---|---|---|---|---|---|---|
| Respiratory rate | >35 | 31~35 | 21~30 | 9~20 | | | <7 |
| (Respiratory | | | | | | | |
| rate/min) | | | | | | | |
| SpO2 (%) | <85 | 85~89 | 90~92 | >92 | | | |
| Temperature (° C.) | | >38.9 | 38~38.9 | 36~37.9 | 35~35.9 | 34~34.9 | <34 |
| Systolic blood | | >199 | | 100~199 | 80~99 | 70~79 | <70 |
| pressure (mmHg) | | | | | | | |
| Heart rate (bpm) | >129 | 110~129 | 100~109 | 50~99 | 40~49 | 30~39 | <30 |
| AVPU | | | | Alert | Negative | Pain | Unconsciousness |

Here, in the content shown in Table 1, “normal range” and “abnormal range” configured from the measurement values of vital signs of a group are adopted as the references. The range configured here can be changed in consideration of the area, age, etc., but basically, the range used as a reference is determined based on the measurement values of the vital signs obtained from the majority of the number of people. The configuration of the reference is the same for respiratory rate, oxygen saturation, blood pressure, and heart rate. Further, in the EWS, scores may be assigned to other parameters such as urine output, oxygen dose flow rate, and pain score as parameters other than the six major vital signs. In the conventional scoring method, the “normal range” and the “abnormal range” configured based on the measurement value of the vital signs of a group become the references. For this reason, it is difficult to say that the detection takes into account the intra-individual variation of the subject.

In other words, the reference configured from the measurement values of the vital signs of a group cannot correspond to the individual characteristics in the vital signs. For example, in young people and elderly people, the body temperatures in a calm state, or the temperature fluctuations in one day are very different. Moreover, the value of the vital sign varies greatly between subjects, depending on the presence or absence of a disease state such as hypertension.

That is, in consideration of the age of the subject or the presence or absence of a disease state, the “normal range” or “abnormal range” configured from the measurement values of the vital signs of a group may not be an appropriate reference. Therefore, the application of the “vital sign abnormal value detection” technology in the present disclosure to the “vital scoring” technology was also studied.

From the content described above, for the abnormality determination and scoring determination of vital signs, the inventor took into account the property that the vital signs of an individual's body temperature, blood pressure, pulse, and pulse pressure are always normally distributed except for special diseases, and furthermore, performed examination for reducing measure points required for determination while maintaining accuracy of the determination in order to enable rapid determination with a small number of data or a short period of time.

The present disclosure was made in view of the above points, and the present disclosure relates to software, a health condition determination device, and a health condition determination method which reflect vital signs or daily conditions in consideration of the individual differences of the subjects, enable the subjects to determine the intra-individual variation more quickly with high accuracy, and contribute to the subject's health care or the provision of medical care for the characteristics of each individual.

### Solution to Problem

In order to achieve the above purpose, software of the present disclosure is software for determining the health condition of an individual, based on vital information that is a value of a measured vital sign, the software configured to be installed on an information processing device including: an information input means for receiving an input of information on measurement date and time and vital information measured from the same individual and following a normal distribution; an information recording means for recording the input vital information and information on measurement date and time; a reference calculation means for calculating at least one selected from a mean μ and a standard deviation σ of all or part of the multiple pieces of recorded vital information; and a determination means for determining whether the input predetermined vital information is an abnormal value, based on a predetermined numerical range configured based on the at least one selected from the mean μ and the standard deviation σ, wherein the predetermined numerical range is established using the vital information including at least 4 pieces recorded in the information recording means and has a value of Equation (1) below as a lower limit, a value of Equation (2) below as an upper limit, and at least one of the lower limit or the upper limit as a reference, Equation (1) and Equation (2) being expressed using the mean μ, the standard deviation σ, n and m that are numbers greater than 0 (zero):

μ−nσ Equation (1); and

μ+mσ Equation (2).

In addition, the software in the specification is a program related to the operation of a computer. The program is made up of a numbered sequence of instructions suitable for processing by a computer.

Here, the information input means may receive the input of the vital information measured from the same individual and following a normal distribution and may record the input vital information in the information recording means, so that the vital information of the same individual can be accumulated. In addition, the same individual as used herein refers to the object to be determined which is determined as to whether the value of the measured vital sign is an abnormal value.

In addition, “vital information following a normal distribution” as used herein means a property that data is normally distributed when a predetermined number of data (for example, 30 pieces of data disclosed in patent document 3) is prepared. Therefore, terms including only “vital information including at least 4 pieces”, which will be described later, do not mean that the data is normally distributed (representing normality).

In addition, the term “individual” as used herein means a single organism (human or animal). In addition, the present disclosure is single software and includes an aspect of recording the vital information of a single identical individual, and an aspect of recording multiple pieces of vital information of the same individual for each same individual. The same individual refers to the same person, for human being, for example.

In addition, the term “the vital information measured from the same individual” as used herein means that the individual can be distinguished at the stage of input by the information input means. For example, it is also possible to distinguish an individual by different formats of input, such as an aspect in which one subject inputs personal vital information, or an aspect in which a specific personal input screen is displayed when the subject deals with information of a plurality of subjects, and inputs the vital information, or the like.

In addition, the information input means receives the input of the vital information measured from the same individual and the information on measurement date and time and records the input vital information and information on measurement date and time in the information recording means, so that the vital information of the same individual is accumulated with the information on measurement date and time. That is, the multiple pieces of vital information of the same individual can be handled in association with the measured data and time. In addition, when comparing with other vital information, it is possible to confirm the situation of displacement and the displacement amount between the vital information to be compared. The information on measurement date and time includes an aspect in which the inputter inputs information on measurement date and time when inputting vital information in the information input means, or an aspect in which the time at which the vital information is input is automatically input in the information input means.

In addition, the reference calculation means calculates the mean μ of all or a part of the multiple pieces of recorded vital information, so that the information of the mean value of the vital information reflecting the variation in the individual of the same individual can be used. In addition, the mean μ as used herein means the value obtained by dividing “the total value of each vital sign” by “the number of data of measurement values of the vital signs”. In addition, the term “the mean μ of multiple pieces of recorded vital information” as used herein includes not only that calculated from the entire data of the recorded vital information but also that calculated from a part of the entire data. Furthermore, the vital information that is the basis for calculating the mean μ may be calculated from continuous data, for example, measured continuously such as every second, every minute, every hour, every day, as well as data extracted at intervals of seconds, minutes, hours, and dates.

Further, the reference calculation means calculates the standard deviation σ of all or part of multiple pieces of recorded vital information so that it is possible to use information of the standard deviation of the vital information reflecting variation in the individual of the same individual. In addition, the standard deviation σ as used herein is “square mean of the deviation” of the vital information of a predetermined condition. More specifically, the “deviation” is a value obtained by subtracting the “mean value of measurement values of the vital signs under a predetermined condition” from “the measurement values of each vital signs” of the vital information under a predetermined condition. The “standard deviation σ of multiple pieces of recorded vital information” includes not only that calculated from the entire data of the recorded vital information but also that calculated from a part of the entire data. Further, the vital information that is the basis for calculating the standard deviation σ may be calculated from not only continuous data such as data measured continuously such as every second, every minute, every hour, or every day, but also data extracted at intervals of seconds, minutes, hours, and dates.

In addition, the reference calculation means calculates at least one selected from the mean μ and the standard deviation σ of all or part of the multiple pieces of recorded vital information, so that it is possible to calculate any one of the mean μ and the standard deviation σ. Further, it is also possible to calculate both the mean μ and the standard deviation σ.

Further, the determination means determines whether the input predetermined vital information is an abnormal value, based on a predetermined numerical range configured based on at least one selected from the mean μ and the standard deviation σ, and thus has a reference in which the intra-individual variation of the same individual is reflected and can determine whether the vital information of the same individual is an abnormal value. That is, the predetermined numerical range which is a determination reference is configured using a mean value or a standard deviation calculated from the vital information obtained for the same individual and therefore is unique to the same individual, and it is possible to determine whether it is abnormal with a reference in which the mean value of the vital information or the dispersion from the mean value is reflected. In addition, “input predetermined vital information” as used herein means vital information to be determined. In addition, the term “predetermined numerical range” referred to herein includes both numerical range configured from previous vital information without including the input predetermined vital information, i.e., the input predetermined vital information to be determined, and numerical range configured to include the predetermined vital information to be determined. In addition, the term “predetermined numerical range” includes both an aspect in which the numerical value to be determined is determined to be “abnormal” when a reference value, for example, the upper limit value is configured and the numerical value is equal to or higher than the upper limit value, and an aspect in which the numerical value is determined to be “abnormal” when the numerical value to be determined exceeds the upper limit. The input predetermined vital information may be recently input vital information. In addition, the input predetermined vital information may be one or more pieces of vital information among previously input vital information.

In addition, the predetermined numerical range is established using the vital information including at least 4 pieces recorded in the information recording means and has a value of Equation (1) below as a lower limit, a value of Equation (2) below as an upper limit, and at least one of the lower limit or the upper limit as a reference, Equation (1) and Equation (2) being expressed using the mean μ, the standard deviation σ, n and m that are numbers greater than 0 (zero), so that it is possible to determine whether the input predetermined vital information is abnormal value, based on the numerical value which is separated by the value of nσ in the negative direction from the mean μ as the lower limit, and the numerical value which is separated by the value of mσ from the mean μ as an upper limit:

μ−nσ Equation (1); and

μ+mσ Equation (2).

In other words, it is possible to determine whether the measurement values of vital signs measured from the same individual is an abnormal value, based on at least one of a value obtained as the lower limit by subtracting nσ from the mean μ and a value obtained as an upper limit by adding mσ to the mean μ. In addition, a reference can be established using the vital information including at least 4 pieces, and thus the quick determination is possible. Furthermore, the values of n and m may be any number greater than 0 as described above and the values of n and m can be appropriately configured in consideration of various conditions such as the strictness of the standard, the type of vital signs, and the history of the subject. In addition, since it is “at least one of a lower limit and an upper limit,” it includes not only an aspect in which only a lower limit or only an upper limit is configured as a reference but an aspect in which both a lower limit and an upper limit are adopted as a reference.

Here, the possibility of using “vital information including at least 4 pieces (measured data)” for abnormality determination (or scoring determination) in the present disclosure will be described in detail. More specifically, it will be explained that if at least 4 pieces of measurement data for vital signs of body temperature, pulse, blood pressure, and pulse pressure are acquired, a determination reflecting the variation in the individual of the subject is possible.

The inventors have confirmed that, based on previous studies, if at least 30 pieces of measurement data can be acquired for the vital data obtained from the same individual, the measurement data reflects the variation within the individual for each subject and is normally distributed (see patent document 3).

For example, as shown in FIGS. 15 to 22, when the pulse was measured under each condition, if 30 pieces of measurement data were prepared, it is possible to obtain normal distribution curves different for each subject based on the measured data. FIGS. 15, 17, 19, and 21 are the results of the pulses obtained from the same subject (herein, referred to as A), and FIGS. 16, 18, 20, and 22 are result of a pulse obtained from other identical subjects (herein, referred to as B). In addition, although 30 pieces of measurement data are shown in FIGS. 15 to 22 and the circle on a curve corresponds to a piece of measurement data, since there exist multiple pieces of overlapping data centered on a mean value, 30 circles do not appear in the figures.

More specifically, FIGS. 15 and 16 are graphs based on the results obtained by measuring pulses every minute and acquiring measurement data of 30 pulses. Results were obtained in which all showed the shape of the normal distribution with the mean value as the vertex. Moreover, in the cases of A and B, the mean values used as the vertex differ, and the values located at both ends of a curve (minimum value and maximum value) also differ. Thus, it is obvious that a normal distribution of each individual is obtained. This point is also confirmed in the same tendency in FIGS. 17 to 22.

In addition, FIGS. 17 and 18 are graphs based on the results obtained by measuring pulses every 7 minutes and obtaining measurement data for 30 pulses. Thus, even when the time interval to measure was changed, the shape of the normal distribution which made the mean value of each subject the vertex was obtained.

In addition, FIGS. 19 and 20 are graphs based on the results of acquiring measurement data of 30 pieces of pulses at irregular times during the day. Furthermore, FIG. 21 is a graph based on the result of obtaining the measurement data of 30 pieces of pulses at an irregular time for 30 hours, and FIG. 22 is a graph based on the result of obtaining the measurement data of 30 pieces of pulses at an irregular time for 30 days. As shown here, it was confirmed that if 30 pieces of measurement data were acquired even if the data was not regularly acquired at regular intervals, the data took the form of a normal distribution with the mean value of each subject as the vertex.

In addition, for example, as shown in FIGS. 23 and 24, when the 30 pieces of measurement data for body temperature were also prepared, the normal distribution curve different for each subject was obtained based on the measurement data. FIGS. 23 and 24 are graphs based on the results obtained by measuring body temperature every 2 minutes to acquire 30 pieces of measurement data of body temperature. In addition, in FIGS. 23 and 24, the subjects who were measured for the body temperature were different. Thus, even in the case of body temperature, it was confirmed that if the 30 pieces of measurement data were acquired, the data took the shape of the normal distribution with the mean value of each subject as the vertex.

The point that the normal distribution different for each individual can be obtained from 30 pieces of measurement data was confirmed not only for pulse and body temperature but also for blood pressure (systolic blood pressure and diastolic blood pressure) and pulse pressure.

Therefore, the inventors have found that a normal distribution reflecting the variation within the individual of the subject can be obtained by obtaining at least 30 pieces of measurement data for the vital signs regardless of the length of time or the regularity of the measurement interval and that the 30 pieces of measurement data can be used as a biomarker.

Here, the inventors performed a statistical hypothesis test between the data group for 4 days and the data group for 30 days for the value of vital signs measured once a day. As a result, there was no significant difference (P>012.05) even through comparison between the data group for 4 days and the data group for 30 days, and the inventor proved that there is no difference (P is close to 1) to verify that there is no difference in detection accuracy between the data group for 4 days and the data group for 30 days.

In more detail, measurement is performed once a day for each vital sign of systolic blood pressure, diastolic blood pressure, pulse pressure, pulse, and body temperature, and the “mean value” and “standard deviation” of each data group were calculated with respect to a data group for 4 days (4 day-data group), a data group for 10 days (10 day-data group), a data group for 14 days (14-day data group), and a data group for 30 days (30-day data group). Also, the number of data is based on 156 persons (n=156).

To compare the data groups of the 4-day data group, the 10-day data group, the 14-day data group, and the 30-day data group, the P value of each vital sign was calculated based on one-way ANOVA, which is a type of analysis of variance (ANOVA). In addition, the P value is a significance probability that measures the evidence for rejecting the null hypothesis. In this test, the closer the P value is to 1, the less significant difference in each data group is assumed. Table 2 shows the results of the mean value, standard deviation, and P value of the data group.

In addition, one-way ANOVA can be calculated by the conventional method, so detailed description is omitted, but for each data group, it can be calculated by calculating the within-group sum of squares, inter-group sum of squares, degrees of freedom, F-value, and P-value in stages.

TABLE 2

| | 4 day-data group | | 10 day-data group | | 14 day-data group | | 30 day-data group | | |
|---|---|---|---|---|---|---|---|---|---|
| | Mean | standard | Mean | standard | Mean | standard | Mean | standard | P |
| | value | deviation | value | deviation | value | deviation | value | deviation | value |
| Systolic | 120.00 | 16.01 | 120.26 | 14.64 | 120.18 | 14.41 | 120.15 | 13.51 | 0.999 |
| blood | | | | | | | | | |
| pressure | | | | | | | | | |
| Diastolic | 70.84 | 8.92 | 70.90 | 7.90 | 70.85 | 7.72 | 70.72 | 7.30 | 0.998 |
| blood | | | | | | | | | |
| pressure | | | | | | | | | |
| Pulse | 49.16 | 12.73 | 49.40 | 11.47 | 49.36 | 11.39 | 49.44 | 10.72 | 0.997 |
| pressure | | | | | | | | | |
| Pulse | 70.51 | 9.86 | 70.78 | 9.37 | 70.90 | 9.31 | 70.68 | 9.10 | 0.987 |
| Body | 36.47 | 0.28 | 36.48 | 0.21 | 36.47 | 0.21 | 36.47 | 0.20 | 0.960 |
| temperature | | | | | | | | | |

As shown in Table 2, the P values for the 4 data groups of the 4-day data group, 10-day data group, 14-day data group, and 30-day data group are 0.960 to 0.999 which is a value close to 1 with respect to each vital sign of systolic blood pressure, diastolic blood pressure, pulse pressure, pulse, and body temperature. Through the results, it is possible to assume that there is no significant difference between the data groups. Moreover, even when the mean value and standard deviation of each data group were compared, the difference of the mean value and the difference of the standard deviation between the data groups were very small values. From the above results, for each vital sign, the mean value and standard deviation of the data group for 4 days compared with the mean value and standard deviation of the data group for 30 days were not significantly different (P>0.05), and it was also confirmed that it is possible to assume that there was no difference.

Then, accordingly, the present disclosure is based on the ideas that when performing abnormality determination or scoring determination of the vital sign of the individual subject, the mean value (μ) and standard deviation (σ) based on the vital information including at least 4 pieces can be employed as the calculation basis of the determination reference (predetermined numerical range).

That is, the determination reference configured from the mean value (μ) and standard deviation (σ) based on the vital information including at least 4 pieces also reflects the intra-individual variation of the individual subject, and quick determination can be performed using the vital information including at least 4 pieces for the determination.

In addition, when the reference calculation means calculates the mean μ and standard deviation σ from vital information measured twice or more per day and for at least 2 days or more, which is recorded in the information recording means, the mean value and standard deviation reflecting intra-individual variation of the same individual can be calculated based on the vital information for at least 2 days. In addition, the vital information measured twice or more per day and for at least 2 days or more, referred to herein, also includes vital information for 2 days or more in total, based on twice a day measurement, for information with intervals in the number of days as well as consecutive days. In addition, the information measured twice a day means information measured once in the morning and once in the afternoon, for example.

In addition, when the reference calculation means calculates the mean μ and standard deviation σ from vital information for at least 4 days or more, which is recorded in the information recording means, the mean value and standard deviation reflecting intra-individual variation of the same individual can be calculated based on the vital information for at least 4 days. As a result, it is possible to increase the accuracy of the reference for determining whether the value is an abnormal value. In addition, the vital information for at least 4 days or more, referred to herein, also includes vital information for 4 days or more in total, for information with intervals in the number of days as well as consecutive days. In addition, vital information for 4 days or more means information in which vital information measured once a day, for example, is prepared for 4 days or more.

In addition, when the vital information includes at least one measurement value of body temperature, pulse, blood pressure, or pulse pressure, determination on whether the vital information is abnormal can be performed while having a determination reference reflecting intra-individual variation for the body temperature, pulse, blood pressure, and pulse pressure.

In addition, when the predetermined numerical range is configured to include vital information determined by the determination means to be an abnormal value, intra-individual variation can be identified, including the state in which the abnormality occurred in the value of the vital sign of the subject to enable abnormality determination for the vital signs.

In addition, when the predetermined numerical range is configured to exclude vital information determined by the determination means to be an abnormal value, vital information that is not stable under special circumstances as follows is not included in the calculation basis of the determination reference, and thus the accuracy of determination can be increased. Vital information that is not stable under special circumstances as used herein means, for example, a value of vital signs measured at the time of medical intervention for a subject, that is, immediately after the subject is hospitalized by a doctor's diagnosis (instruction). The value of vital signs measured under such circumstances can easily have an unstable value in view of intra-individual variation of the subject's vital signs, so this value is excluded from the calculation basis of the determination reference.

In addition, when the predetermined numerical range is configured to exclude the input predetermined vital information, the determination reference of abnormality for the vital signs is provided without including the value of the vital sign to be determined. According to this, when the value of the vital sign to be determined becomes a numerical value that can be considered abnormal in view of the change in the subject's vital signs (for example, the body temperature has risen), the numerical value considered to be abnormal is excluded from the calculation basis of the determination reference to increase the accuracy of the determination for the presence or absence of abnormal vital sings.

In addition, when the predetermined numerical range is configured including the input predetermined vital information, the value of the vital sign to be determined is included to provide the abnormality determination reference of vital signs. According to this, the number of data serving as the reference for determining abnormality for vital signs increases, and thus it becomes possible to prepare the determination reference further reflecting the tendency of the intra-individual variation of a subject.

In addition, when the predetermined numerical range is configured to exclude the vital information measured from the subject in a predetermined state, a reference for determining abnormality for vital signs is prepared excluding the value of vital signs measured under a special state in which the subject's vital signs are not stable. That is, for example, it is an aspect in which the value of the body temperature measured in a state in which the subject has taken an antipyretic agent and the body temperature is not stable (it does not show the original tendency to change) is excluded from the calculation basis of the determination reference. Thereby, the accuracy of abnormality determination of vital signs for a short period of time can be increased. In addition, the predetermined state referred to herein refers to a special state in which the vital signs of the subject are not stable, and the content thereof is not limited to body temperature at the time of taking the antipyretic agent. For example, the predetermined state includes a state in which a subject takes drugs that act on blood pressure or pulse or has been given a prescription or treatment that acts on changes in other vital signs.

In addition, the reference calculation means further calculates, from the vital information recorded in the information recording means, a control vital mean value that is a mean value of the last vital mean value, which is the mean value of vital signs for the last 4 days, and the mean value of vital signs for the last 30 days, the software further includes software for causing the information processing device to function as the second determination means, and in case that the second determination means determines a value as a condition deterioration tendency value when the difference between the recent vital mean value and the control vital mean value exceeds a predetermined range, information that predicts deterioration of the health condition of the same individual can be provided based on the change in the mean vital value. In other words, based on the changes in the mean vital value for the last 4 days and the last 1 month, a phenomenon in which the vital mean value itself changes significantly is recognized as a large change in the health condition of the same subject and is used as prediction information causing abnormality. In addition, for “last” here, both the aspect including vital information which is the object to be determined and the aspect which does not include the vital information are employable.

In addition, when the information input means receives the input of the re-measured vital information and the information on measurement date and time of the same individual measured again after the determination means determines that the input predetermined vital information is an abnormal value, the vital information of the same individual that has been re-measured is further recorded in addition to the vital information used as the basis for the determination. For example, in a case where the value of the vital information that was the basis of the determination becomes an erroneous value for some reason, such as a bad measurement method, and the measured value is determined to be an abnormal value, inputting and recording of vital information to identify whether the determination result is correct may be performed again.

When the determination means determines whether the re-measured vital information is an abnormal value, determination on whether the re-measured vital information is an abnormal value may be performed. That is, for example, as described above, when the value of vital information, which is the basis for the first determination as an abnormal value, is an incorrect value for some reason, determination on the presence or absence of abnormality may be performed again. Furthermore, in this case, by using the re-measured vital information, the mean value and standard deviation to be used for the next determination, and the determination reference configured based on the same may also be established.

Further, when the information recording means can record the individual identification information that can identify the individual in association with the vital information, each vital information is identified and handled for each individual. That is, for example, by managing the vital information of a plurality of subjects with single software, it becomes possible to determine the vital information of a plurality of subjects.

In addition, when the vital sign is a vital sign measured from at least one of humans and animals, it is possible to configure a human or an animal as an object to be determined. In addition, the animal referred to herein is not particularly limited in kind, and any animal, the vital sign value of which can be measured, can be an object to be determined for abnormality.

In order to achieve the above purpose, the health condition determination device of the present disclosure is a health condition determination device for determining a health condition of an individual, based on vital information that is a value of a measured vital sign and includes: an information input means for receiving an input of information on measurement date and time and vital information measured from the same individual and following a normal distribution; an information recording means for recording the input vital information and information on measurement date and time; a reference calculation means for calculating at least one selected from a mean μ and a standard deviation σ of all or part of the multiple pieces of recorded vital information; a determination means for determining whether the input predetermined vital information is an abnormal value, based on a predetermined numerical range configured based on the at least one selected from the mean μ and the standard deviation σ; and a display means capable of displaying the determination result determined by the determination means, wherein the predetermined numerical range is established using the vital information including at least 4 pieces recorded in the information recording means and has a value of Equation (1) below as a lower limit, a value of Equation (2) below as an upper limit, and at least one of the lower limit or the upper limit as a reference, Equation (1) and Equation (2) being expressed using the mean μ, the standard deviation σ, n and m that are numbers greater than 0 (zero):

μ−nσ Equation (1); and

μ+mσ Equation (2).

Here, the predetermined numerical range is established using the vital information including at least 4 pieces recorded in the information recording means, and when a value of Equation (1) below and a value of Equation (2) below, which are expressed using the mean μ, the standard deviation ρ, n and m that are numbers greater than 0 (zero), are regarded as a lower limit and as an upper limit, respectively, and at least one of the lower limit or the upper limit is used as a reference, it is possible to determine whether the input predetermined vital information is abnormal value, based on the numerical value which is separated by the value of nσ in the negative direction from the mean μ as the lower limit, and the numerical value which is separated by the value of mσ from the mean μ as an upper limit:

μ−nσ Equation (1); and

μ+mσ Equation (2).

In other words, it is possible to determine whether the measurement values of vital signs measured from the same individual is an abnormal value, based on at least one of a value obtained as the lower limit by subtracting nσ from the mean μ and a value obtained as an upper limit by adding mσ to the mean μ. In addition, a reference can be established using the vital information including at least 4 pieces and thus the quick determination is possible.

Moreover, the determination result can be displayed by the display means capable of displaying the determination result determined by the determination means and be confirmed.

Further, in order to achieve the purpose, a health condition determination method of the present disclosure, which is a method executed by a computer and is configured to determine a health condition of an individual, based on vital information which is measured values of vital signs, includes: a reference calculating step of calculating at least one selected from a mean μ and a standard deviation σ of a predetermined number of vital information or more among vital information measured from the same individual and following a normal distribution; and a determination step of determining whether input predetermined vital information is an abnormal value, based on a predetermined numerical range configured based on the at least one selected from the mean μ and the standard deviation σ, wherein the predetermined numerical range is established using the vital information including at least 4 pieces and has a value of Equation (1) below as a lower limit, a value of Equation (2) below as an upper limit, and at least one of the lower limit or the upper limit as a reference, Equation (1) and Equation (2) being expressed using the mean μ, the standard deviation σ, n and m that are numbers greater than 0 (zero).

Here, in the reference calculating process, by calculating at least one selected from a mean μ and a standard deviation σ of a predetermined number of vital information or more among vital information measured from the same individual, it is possible to use information of a mean and a standard deviation of the vital information reflecting the intra-individual variation of the same individual.

In addition, the predetermined numerical range is established using the vital information including at least 4 pieces recorded in the information recording means, and when a value of Equation (1) below and a value of Equation (2) below, which are expressed using the mean μ, the standard deviation σ, n and m that are numbers greater than 0 (zero), are regarded as a lower limit and as an upper limit, respectively, and at least one of the lower limit or the upper limit is used as a reference, it is possible to determine whether the input predetermined vital information is abnormal value, based on the numerical value which is separated by the value of nσ in the negative direction from the mean μ as the lower limit, and the numerical value which is separated by the value of mσ from the mean μ as an upper limit:

μ−nσ Equation (1); and

μ+mσ Equation (2).

In other words, it is possible to determine whether the measurement values of vital signs measured from the same individual is an abnormal value, based on at least one of a value obtained as the lower limit by subtracting nσ from the mean μ and a value obtained as an upper limit by adding mσ to the mean μ. In addition, a reference can be established using the vital information including at least 4 pieces and thus the quick determination is possible.

In order to achieve the above purpose, software of the present disclosure is software for scoring vital information which is information related to obtained vital signs to determine a health condition of an individual, based on the obtained score result information, the software to be installed on an information processing device including: an information input means for receiving an input of information on acquisition date and time and vital information obtained from the same individual and following a normal distribution; an information recording means for recording the input vital information and information on acquisition date and time; a reference calculation means for calculating a mean μ and a standard deviation σ of all or part of the multiple pieces of recorded vital information; a scoring processing means for scoring input predetermined vital information to calculate score result information which is a value of a score, based on a predetermined scoring condition; and a score determination means for determining whether the score result information is an abnormal value, based on a predetermined score determination condition, wherein the vital information includes at least one measurement value of body temperature, pulse, blood pressure, or pulse pressure, and the predetermined scoring condition, and the predetermined scoring condition, for at least one measurement value of body temperature, pulse, blood pressure, or pulse pressure, is established using the vital information including at least 4 pieces, has a value of Equation (1) below as a lower limit and a value of Equation (2) below as an upper limit, which are expressed using the mean μ, the standard deviation σ, n and m that are numbers greater than 0 (zero), and has at least one of the lower limit or the upper limit as a reference:

μ−nσ Equation (1); and

μ+mσ Equation (2).

In addition, the software in the specification is a program related to the operation of a computer. The program is also made up of a numbered sequence of instructions suitable for processing by a computer.

Here, the information input means may receive the input of the vital information measured from the same individual and following a normal distribution and may record the input vital information in the information recording means, so that the vital information of the same individual can be accumulated. In addition, the same individual as used herein refers to the object to be determined on which scoring is performed based on the value of the measured vital sign.

In addition, “vital information following a normal distribution” as used herein means a property that data is normally distributed when a predetermined number of data (for example, 30 pieces of data disclosed in patent document 3) is prepared. Therefore, terms including only “vital information including at least 4 pieces”, which will be described later, do not mean that the data is normally distributed (representing normality).

In addition, the information input means receives the input of the vital information acquired from the same object and information on acquisition date and time, and records the input vital information and information on acquisition date and time in the information recording means, so that the vital information of the same individual is accumulated with the information on acquisition date and time. That is, the multiple pieces of vital information of the same individual can be handled in association with the information on acquisition date and time. In addition, when comparing with other vital information, it is possible to confirm the situation of displacement and the displacement amount between the vital information to be compared. The information on acquisition date and time herein includes an aspect in which the inputter inputs information on acquisition date and time when inputting vital information in the information input means, or an aspect in which the time at which the vital information is input is automatically input in the information input means. The information on acquisition date and time includes the date and time when the vital signs were measured or the date and time when the vital signs were evaluated (e.g., consciousness level).

In addition, the reference calculation means calculates the mean μ of all or a part of the multiple pieces of recorded vital information, so that the information of the mean value of the vital information reflecting the variation in the individual of the same individual can be used. In addition, the mean μ as used herein means the value obtained by dividing “the total of the measurement value of each vital sign” by “the number of data of the measurement value of vital sign”. In addition, the term “the mean μ of multiple pieces of recorded vital information” as used herein includes not only that calculated from the entire data of the recorded vital information but also that calculated from a part of the entire data. Furthermore, the vital information that is the basis for calculating the mean p may be calculated from continuous data, for example, measured continuously such as every second, every minute, every hour, every day, as well as data extracted at intervals of seconds, minutes, hours, and dates.

Further, the reference calculation means calculates the standard deviation σ of all or part of multiple pieces of recorded vital information so that it is possible to use information of the standard deviation of the vital information reflecting variation in the individual of the same individual. In addition, the standard deviation σ as used herein is “square mean of the deviation” of the vital information of a predetermined condition. More specifically, the “deviation” is a value obtained by subtracting the “mean value of measurement values of the vital signs under a predetermined condition” from “the measurement values of each vital signs” of the vital information under a predetermined condition. The “standard deviation σ of multiple pieces of recorded vital information” includes not only that calculated from the entire data of the recorded vital information but also that calculated from a part of the entire data. Further, the vital information that is the basis for calculating the standard deviation σ may be calculated from not only continuous data such as data measured continuously such as every second, every minute, every hour, or every day, but also data extracted at intervals of seconds, minutes, hours, and dates.

Further, the scoring processing means scores the input predetermined vital information to calculate the score result information which is the value of the score, based on a predetermined scoring condition, thereby converting the input vital information into score result information (score) according to the content thereof. The “input predetermined vital information” referred to herein means vital information to be scored.

Further, the score determination means determines whether the score result information is an abnormal value, based on a predetermined score determination condition, so that it is possible to determine whether the score result information obtained from the contents of the vital information obtained from the same individual is an abnormal value. In addition, in the determination based on the predetermined score determination conditions herein, there may be an aspect of determining whether the score result information obtained from one vital sign is an abnormal value or an aspect of determining the total points of multiple pieces of score result information, and furthermore, an aspect of determining the combination of two or more pieces of score result information.

In addition, a predetermined scoring condition, for at least one measurement value of body temperature, pulse, blood pressure, or pulse pressure, is established using the vital information including at least 4 pieces and has a value of Equation (1) below as a lower limit, a value of Equation (2) below as an upper limit, and at least one of the lower limit or the upper limit as a reference, Equation (1) and Equation (2) being expressed using the mean μ, the standard deviation σ, n and m that are numbers greater than 0 (zero), so that the score result information according to the content thereof can be obtained based on the numerical value which is separated by the value of nσ in the negative direction from the mean μ as the lower limit and the numerical value which is separated by the value of mσ from the mean μ as an upper limit. In addition, this reference is a reference which reflects the intra-individual variation of the same individual, and enables the vital information of the same individual to be scored in the form that reflects the variation within the individual:

μ−nσ Equation (1); and

μ+mσ Equation (2).

In other words, it is possible to obtain score result information for the measurement values of vital signs measured from the same individual, based on at least one of a value obtained as the lower limit by subtracting nσ from the mean μ and a value obtained as an upper limit by adding mσ to the mean μ. In addition, a reference can be established using the vital information including at least 4 pieces and thus the quick determination is possible. Furthermore, the values of n and m may be any number greater than 0 as described above and the values of n and m can be appropriately configured in consideration of various conditions such as the strictness of the standard, the type of vital signs, and the history of the subject.

In addition, the term “a predetermined scoring condition” as used herein includes both that configured from the previous vital information without including the predetermined vital information to be scored, that is, the input predetermined vital information, and that configured to include predetermined vital information to be scored. In addition, the input predetermined vital information may be recently input vital information. In addition, the input predetermined vital information may be one or more pieces of vital information among previously input vital information. In addition, the “predetermined scoring condition” as used herein includes both an aspect in which, when a reference value, for example, a certain value is configured, a numerical value of targets for scoring becomes two points when it is the certain value or higher and one point when it is less than the certain value, and an aspect in which the numerical value of targets for scoring becomes two points when exceeding the certain value and one point when it is the certain value or lower. In addition, since it is “at least one of a lower limit and an upper limit,” it includes not only an aspect in which only a lower limit or only an upper limit is configured as a reference but an aspect in which both a lower limit and an upper limit are adopted as a reference.

In addition, when the reference calculation means calculates the mean μ and standard deviation σ from vital information measured twice or more per day and for at least 2 days or more, which is recorded in the information recording means, the mean value and standard deviation reflecting intra-individual variation of the same individual can be calculated based on the vital information for at least 2 days. In addition, the vital information measured twice or more per day and for at least 2 days or more, referred to herein, also includes vital information for 2 days or more in total, based on twice a day measurement, for information with intervals in the number of days as well as consecutive days. In addition, the information measured twice a day means information measured once in the morning and once in the afternoon, for example.

In addition, when the reference calculation means calculates the mean μ and standard deviation σ from vital information for at least 4 days or more, which is recorded in the information recording means, the mean value and standard deviation reflecting intra-individual variation of the same individual can be calculated based on the vital information for at least 4 days. As a result, it is possible to increase the accuracy of the reference for calculating the score determination information. In addition, the vital information for at least 4 days or more, referred to herein, also includes vital information for 4 days or more in total, for information with intervals in the number of days as well as consecutive days. In addition, vital information for 4 days or more means information in which vital information measured once a day, for example, is prepared for 4 days or more.

In addition, when the vital information has a measurement value of oxygen saturation, score result information can be obtained for the oxygen saturation measured from the same individual to be determined as to whether the information is an abnormal value.

In addition, when the scoring condition is a predetermined numerical range configured previously for the measurement value of oxygen saturation, if the measurement value of oxygen saturation acquired from the same individual was input as vital information, the score result information corresponding to the content can be obtained based on a predetermined numerical range configured in advance. In addition, the “predetermined numerical range” used herein can adopt the numerical range configured from the measurement values of the vital signs of a group. In addition, the term “predetermined numerical range” used herein includes both an aspect in which, when a reference value, for example, a certain value is configured, a numerical value of targets for scoring becomes two points when it is the certain value or higher and one point when it is less than the certain value, and an aspect in which the numerical value of targets for scoring becomes two points when exceeding the certain value and one point when it is the certain value or lower.

In addition, when the vital information has a consciousness level evaluation result obtained by observing a consciousness level, score result information can be obtained with respect to the consciousness level evaluation result obtained from the same individual to be determined as to whether the information is an abnormal value.

In addition, when the scoring condition is a predetermined observation state indicating the degree of consciousness level with respect to the consciousness level evaluation result, the consciousness level evaluation result obtained from the same individual is applied to the contents of the predetermined observation state and then the score result information according to the content can be obtained. The content of the predetermined observation state is, for example, the content of the AVPU response used for the evaluation of the consciousness level or the content indicating the state of confusion.

Further, when the score determination means determines the score result information as an abnormal value, when the abnormality is determined by dividing the abnormality into at least two stages, the treatment after the determination of the score result information can be diversified. For example, even if the status indicates abnormality, if the numerical value of the score result information is small, it is notified as “caution”, and if the numerical value of the score result information is large, it is notified as “warning”, so that not all abnormalities are treated uniformly. As a result, when the determination is performed, it is possible to efficiently deal with the treatment after the determination such as whether a doctor's check is immediately necessary.

In addition, when the predetermined scoring condition is configured to include vital information that is a calculation basis of the score result information determined as an abnormal value by the score determination means, intra-individual variation can be identified, including the state in which the abnormality occurred in the value of the vital sign of the subject to enable abnormality determination of the vital signs.

In addition, when the predetermined scoring condition is configured to exclude vital information that is a calculation basis of the score result information determined as an abnormal value by the score determination means, vital information that is not stable under special circumstances as follows is not included in the calculation basis of the determination reference, and thus the accuracy of determination can be increased. Vital information that is not stable under special circumstances as used herein means, for example, a value of vital signs measured at the time of medical intervention for a subject, that is, immediately after the subject is hospitalized by a doctor's diagnosis (instruction). The value of vital signs measured under such circumstances can easily have an unstable value in view of intra-individual variation of the subject's vital signs, so this value is excluded from the calculation basis of the determination reference.

In addition, when the predetermined scoring condition is configured to exclude the input predetermined vital information, the scoring condition is provided without including the value of the vital sign to be determined. According to this, when the value of the vital sign to be determined becomes a numerical value that can be considered abnormal in view of the change in the subject's vital signs (for example, the body temperature has risen), the numerical value considered to be abnormal is excluded from the calculation basis of the scoring condition to increase the accuracy of scoring and the accuracy of the determination for the presence or absence of abnormality based on the scoring.

In addition, when the predetermined scoring condition is configured including the input predetermined vital information, the value of the vital signs to be determined is included to provide the scoring condition. According to this, the number of data serving as the basis of scoring condition increases, and thus it becomes possible to prepare the scoring reference further reflecting the tendency of the intra-individual variation of a subject.

In addition, when the predetermined scoring condition is configured to exclude the vital information measured from the subject in a predetermined state, the scoring condition is prepared excluding the value of vital signs measured under a special state in which the subject's vital signs are not stable. That is, for example, it is an aspect in which the value of the body temperature measured in a state in which the subject has taken an antipyretic agent and the body temperature is not stable (it does not show the original tendency to change) is excluded from the calculation basis of the scoring condition. Thereby, the accuracy of the determination of abnormality, based on scoring for a short period of time can be increased. In addition, the predetermined state referred to herein refers to a special state in which the vital signs of the subject are not stable, and the content thereof is not limited to body temperature at the time of taking the antipyretic agent. For example, the predetermined state includes a state in which a subject takes drugs that act on blood pressure or pulse or has been given a prescription or treatment that acts on changes in other vital signs.

Further, in order to achieve the purpose, a health condition determination device of the present disclosure is a health condition determination device for scoring vital information which is information related to obtained vital signs to determine a health condition of an individual, based on the obtained score result information, and is configured to include: an information input means for receiving an input of vital information obtained from the same individual and following a normal distribution and information on acquisition date and time; an information recording means for recording the input vital information and information on acquisition data and time; a reference calculation means for calculating a mean μ and a standard deviation σ of all or a part of multiple recorded vital information; a scoring processing means for scoring the input predetermined vital information to calculate score result information which is a value of a score, based on a predetermined scoring condition; a score determination means for determining whether the score result information is an abnormal value, based on a predetermined score determination condition; and a display means capable of displaying the determination result determined by the score determination means, wherein the vital information includes at least one measurement value of body temperature, pulse, blood pressure, or pulse pressure, and the predetermined scoring condition, for at least one measurement value of body temperature, pulse, blood pressure, or pulse pressure, is established using the vital information including at least 4 pieces, has a value of Equation (1) below as a lower limit and a value of Equation (2) below as an upper limit, which are expressed using the mean μ, the standard deviation σ, n and m that are numbers greater than 0 (zero), and has at least one of the lower limit or the upper limit as a reference:

μ−nσ Equation (1); and

μ+mσ Equation (2).

Here, a predetermined scoring condition, for at least one measurement value of body temperature, pulse, blood pressure, or pulse pressure, is established using the vital information including at least 4 pieces, has a value of Equation (1) below as a lower limit and a value of Equation (2) below as an upper limit, and has at least one of the lower limit or the upper limit as a reference, Equation (1) and Equation (2) being expressed using the mean μ, the standard deviation σ, n and m that are numbers greater than 0 (zero), so that the score result information according to the content thereof can be obtained based on the numerical value which is separated by the value of nσ in the negative direction from the mean μ as the lower limit and the numerical value which is separated by the value of mσ from the mean μ as an upper limit. In addition, this reference is a reference which reflects the intra-individual variation of the same individual, and enables the vital information of the same individual to be scored in the form that reflects the variation within the individual:

μ−nσ Equation (1); and

μ+mσ Equation (2).

In other words, score result information for the measurement values of vital signs measured from the same individual can be obtained based on at least one of a value obtained as the lower limit by subtracting nσ from the mean μ and a value obtained as an upper limit by adding mσ to the mean μ. In addition, a reference can be established using the vital information including at least 4 pieces and thus the quick determination is possible.

Moreover, the determination result can be displayed by the display means capable of displaying the determination result determined by the score determination means and be confirmed.

Further, in order to achieve the purpose, a health condition determination method of the present disclosure, which is a method executed by a computer and is configured to score vital information which is information related to obtained vital signs to determine a health condition of an individual, based on the obtained score result information, includes: an information recording step of receiving and recording an input of vital information obtained from the same individual and following a normal distribution; a reference calculating step of calculating a mean μ and a standard deviation σ of all or a part of multiple recorded vital information; a scoring processing step of scoring the input predetermined vital information to calculate score result information which is a value of a score, based on a predetermined scoring condition; and a score determining step of determining whether the score result information is an abnormal value, based on the predetermined score determination condition, wherein the vital information includes at least one measurement value of body temperature, pulse, blood pressure, or pulse pressure, and the predetermined scoring condition, for at least one measurement value of body temperature, pulse, blood pressure, or pulse pressure, is established using the vital information including at least 4 pieces, has a value of Equation (1) below as a lower limit and a value of Equation (2) below as an upper limit, which are expressed using the mean μ, the standard deviation σ, n and m that are numbers greater than 0 (zero), and has at least one of the lower limit or the upper limit as a reference.

Here, in the reference calculating process, by calculating at least one selected from a mean μ and a standard deviation σ of a predetermined number of vital information or more among vital information measured from the same individual, it is possible to use information of a mean and a standard deviation of the vital information reflecting the intra-individual variation of the same individual.

In addition, a predetermined scoring condition, for at least one measurement value of body temperature, pulse, blood pressure, or pulse pressure, is established using the vital information including at least 4 pieces and has a value of Equation (1) below as a lower limit, a value of Equation (2) below as an upper limit, and at least one of the lower limit or the upper limit as a reference, Equation (1) and Equation (2) being expressed using the mean μ, the standard deviation σ, n and m that are numbers greater than 0 (zero), so that the score result information according to the content thereof can be obtained based on the numerical value which is separated by the value of nσ in the negative direction from the mean μ as the lower limit and the numerical value which is separated by the value of mσ from the mean μ as an upper limit. In addition, this reference is a reference which reflects the intra-individual variation of the same individual, and enables the vital information of the same individual to be scored in the form that reflects the variation within the individual:

μ−nσ Equation (1); and

μ+mσ Equation (2).

In other words, score result information for the measurement values of vital signs measured from the same individual can be obtained based on at least one of a value obtained as the lower limit by subtracting nσ from the mean μ and a value obtained as an upper limit by adding mσ to the mean μ. In addition, a reference can be established using the vital information including at least 4 pieces and thus the quick determination is possible.

### Advantageous Effects of Invention

Software, a health condition determination device, and a health condition determination method according to the present disclosure reflect vital signs or daily conditions in consideration of the individual differences of the subjects, enable the subjects to determine the intra-individual variation more quicky with high accuracy, and contribute to the subject's health care or the provision of medical care for the characteristics of each individual.

### BRIEF DESCRIPTION OF DRAWINGS

FIG. 1 is a view showing a schematic configuration of a tablet terminal incorporating software to which the present disclosure is applied (1 first system configuration);

FIG. 2 is a schematic diagram showing a second system configuration with software to which the present disclosure is applied;

FIG. 3 is a schematic diagram showing a third system configuration with software to which the present disclosure is applied;

FIG. 4 is a block diagram showing the structure of an operation unit, an information transmission/reception unit, and an information recording unit;

FIG. 5 is a schematic diagram showing an example of extraction of vital information;

FIG. 6A is a schematic diagram showing an example of a device used when functioning the software to which the present disclosure is applied, and FIG. 6B is a schematic diagram showing another example of the device;

FIG. 7 is a schematic diagram illustrating an example of an input screen of a value of a vital sign;

FIG. 8 is a schematic diagram illustrating another example of an input screen of a value of a vital sign;

FIG. 9A is a graph of a normal distribution curve created based on vital information of a plurality of subjects, and FIG. 10B is a graph of a normal distribution curve created based on vital information of the same subject;

FIG. 10 is a schematic diagram illustrating an example of a thermal table;

FIG. 11 is a schematic diagram illustrating an example of an image showing a result of scoring in an electronic chart;

FIG. 12 is a schematic diagram showing an example of an image showing a result of scoring by application software used in a smartphone terminal;

FIG. 13 is a flowchart showing the flow of information processing from input of vital information to abnormality determination and display of result information;

FIG. 14 is a flowchart showing the flow of information processing from input of vital information to abnormality determination in score value information and display of result information;

FIG. 15 is a normal distribution curve based on the result of having acquired the measurement data of 30 pulses by measuring a pulse every minute;

FIG. 16 is a normal distribution curve based on the result of having acquired the measurement data of 30 pulses by measuring a pulse every minute;

FIG. 17 is a normal distribution curve based on the result of having acquired the measurement data of 30 pulses by measuring a pulse every 7 minutes;

FIG. 18 is a normal distribution curve based on the result of having acquired the measurement data of 30 pulses by measuring a pulse every 7 minutes;

FIG. 19 is a normal distribution curve based on the results of having acquired the measurement data of 30 pulses at irregular times during the day;

FIG. 20 is a normal distribution curve based on the results of having acquired the measurement data of 30 pulses at irregular times during the day;

FIG. 21 is a normal distribution curve based on the results of having acquired the measurement data of 30 pulses at irregular times for 30 hours;

FIG. 22 is a normal distribution curve based on the results of having acquired the measurement data of 30 pulses at irregular times during 30 days;

FIG. 23 is a normal distribution curve based on the result of having acquired the measurement data of 30 body temperatures by measuring a body temperature every 2 minutes;

FIG. 24 is a normal distribution curve based on the result of having acquired the measurement data of 30 body temperatures by measuring a body temperature every 2 minutes;

FIG. 25 is a schematic diagram showing the result determined to be abnormal in body temperature by having performed determination for abnormality of vital signs based on vital information for 4 days or vital information for 5 days;

FIG. 26 is a schematic diagram showing the result determined to be normal (no abnormality) in body temperature by having performed determination for abnormality of vital signs based on vital information for 4 days or vital information for 5 days;

FIG. 27 is a schematic diagram showing the result determined to be abnormal in pulse by having performed determination for abnormality of vital signs based on vital information for 4 days or vital information for 5 days; and

FIG. 28 is a schematic diagram showing the result determined to be normal (no abnormality) in pulse by having performed determination for abnormality of vital signs based on vital information for 4 days or vital information for 5 days.

### BEST MODE FOR CARRYING OUT THE INVENTION

Hereinafter, embodiments of the present disclosure will be described with reference to the drawings and an understanding of the present disclosure will be provided.

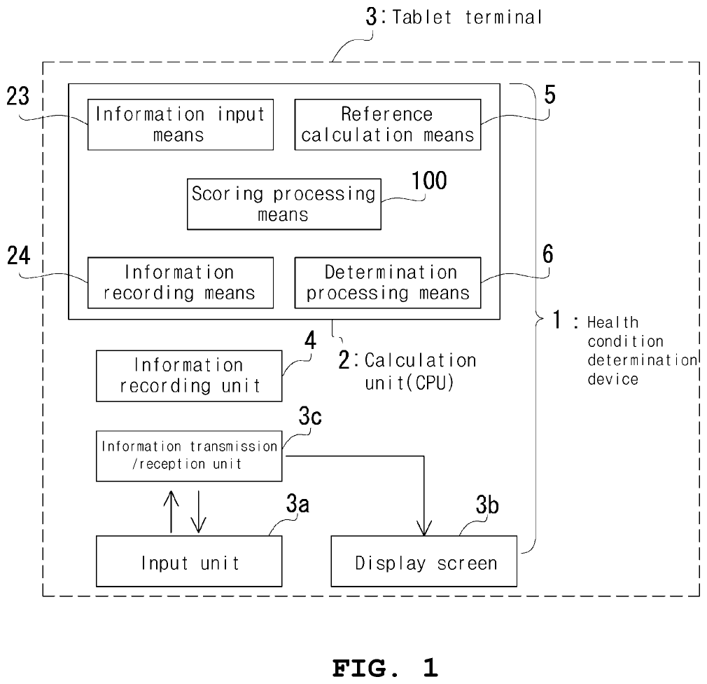

FIG. 1 is a view showing a schematic configuration of a tablet terminal incorporating software to which the present disclosure is applied. In addition, the structure shown below is an example of the present disclosure, and the content of the present disclosure is not limited thereto.

### [1. About Whole Device Configuration]

The software to which the present disclosure is applied can be introduced into a general-purpose information processing device, and provides each information processing function necessary for implementing the present disclosure to the mounted information processing device. As a result, in the tablet terminal 3, it is possible to input the vital information of the subject to determine a health condition reflecting the intra-individual variation in the value of the vital signs of the subject. Further, it is possible to input the vital information of the subject to perform scoring according to the content thereof, and determine whether the obtained score result information (hereinafter, referred to as “score value information”) is an abnormal value.

Further, the information processing device includes a calculation unit such as a CPU, a recording unit such as a RAM or a ROM, an input unit such as a display screen such as a liquid crystal screen or a keyboard, a communication unit for controlling communication with the Internet, and the like. For example, the information processing device is a general purpose personal computer, a tablet terminal, a smart phone, or the like. In addition, as the information processing device, for example, various health care devices, or a medical system or a nursing system installed in a hospital, a facility, or the like may also be covered, and software to which the present disclosure is applied may be mounted and used.

The software to which the present disclosure is applied is downloaded and mounted on the tablet terminal 3 as application software, and a tablet terminal having a function of determining a health condition is used as a health condition determination device 1. The health condition determination device 1, which is an example of a health condition determination device to which the present disclosure is applied, is a device that analyzes short-term individual vital signs of four measured values to determine abnormalities in the health condition. In addition, hereinafter, a user of the health condition determination device 1, that is, a person whose health condition is to be determined, is called a “subject.”

As shown in FIG. 1, the health condition determination device 1 (tablet terminal 3) has a calculation unit 2. The calculation unit 2 is a processing unit which performs each information processing function which the health condition determination device 1 has. In other words, in the software to which the present disclosure is applied, the calculation unit 2 of the tablet terminal 3 functions as an information input means 23, an information recording means 24, a reference calculation means 5, a scoring processing means 100, a determination processing means 6, or the like. The processing function of each of these means allows the transmission and reception of information, recording of information, abnormality determination regarding values of vital signs, configuring of abnormality determination reference in values of vital signs, notification of abnormality determination results regarding values of vital signs, scoring based on the contents of vital information, configuring of scoring conditions (scoring reference information), abnormality determination in score value information, configuring of abnormality determination reference regarding score value, notification of determination result regarding score value, establishing and displaying of display information, or the like.

In addition, the tablet terminal 3 can access an external server, a terminal, and the like through the Internet, and can transmit and receive information between an external server, a terminal, and the like. The information recording means 24, the reference calculation means 5, the scoring processing means 100, and the determination processing means 6 are respectively examples of an “information recording means”, a “reference calculation means”, a “scoring processing means”, and a “determination means (or score determination means) of the claims herein.”

The tablet terminal 3 has an information recording unit 4, an information transmission/reception unit 3c, an input unit 3a, and a display screen 3b.

The information transmission/reception unit 3c is a part that is responsible for transmitting and receiving information between the calculation unit 2, the information recording unit 4, the input unit 3a, the display screen 3b, and the like. In addition, the information transmission/reception unit 3c may be configured to enable the transmission and reception of information between the tablet terminal 3 and the external terminal.

Here, hereinafter, each piece of information handled by the software to which the present disclosure is applied does not necessarily need to be recorded in the information recording unit 4 of the tablet terminal 3. For example, an aspect of the present disclosure may include transmitting and recording various information to an external server or an external terminal through the information transmission/reception unit 3c of the tablet terminal 3, and receiving necessary information from the external server or the like when performing determination or the like.

More specifically, it is not necessary for all of the main components of the health condition determination device 1 to be downloaded to the tablet terminal 3. For example, the tablet terminal 3 may only display information such as determination result information, a normal distribution curve, or a thermal table, and recording and determination processing of various information may be performed by an external server.

The software to which the present disclosure is applied may have a plurality of variations in configuration of the system. Examples of some variations are described below.

### (First System Configuration)

In a schematic configuration of the tablet terminal 3 shown in FIG. 1, the software to which the present disclosure is applied is introduced into the terminal so that the terminal itself can input, record, and determine vital information, display determination results, and configure the determination calculation reference. That is, the device itself can perform the functions of the present disclosure. The schematic configuration shown in FIG. 1 shows the use of the software to which the present disclosure is applied in a device of the “stand alone type” not connected to the Internet environment. The software of the present disclosure can be introduced into an information processing device that is not connected to the Internet environment, for example, various healthcare devices, medical systems/nursing systems such as hospitals, and used as a dedicated device. In addition, since the tablet terminal 3 is exemplified of an information processing device, the connection with the Internet environment is possible. However, the configuration shown in FIG. 1 enables the determination of health condition only by the internal function of the tablet terminal 3.

### (Second System Configuration)

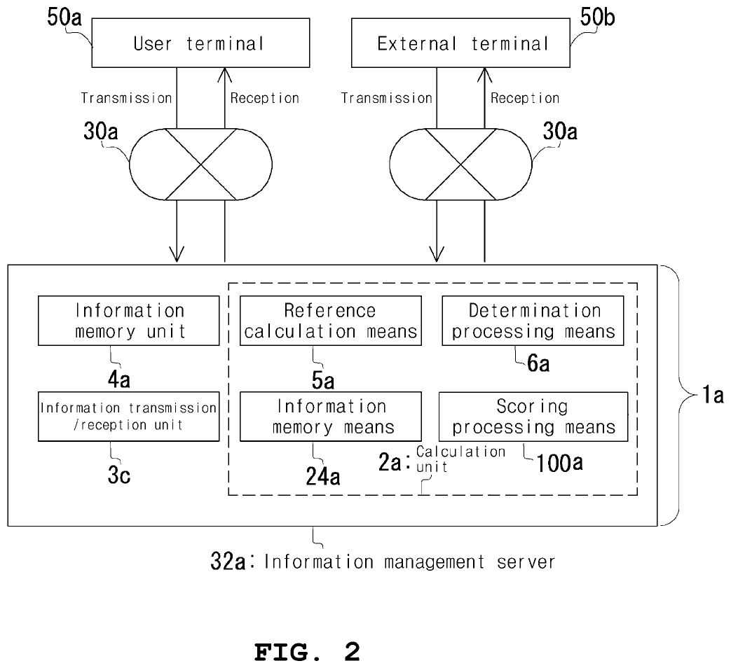

In FIG. 2, a structure which gives an external server the function of the software 1a to which the present disclosure is applied can also be adopted as a second system structure. Here, a user terminal 50a or an external terminal 50b can access the information management server 32a via the Internet 30a. The information management server 32a is an external server provided in a cloud form, for example, and can use the function of the software 1a to which the present disclosure is applied on the information management server 32a.

The information management server 32a has an information recording unit 4a, an information transmission/reception unit 3c, and a calculation unit 2. Moreover, the calculation unit 2 has a reference calculation means 5a, and a determination processing means 6a. The input of the vital information is performed through the user terminal 50a or the external terminal 50b, and information input from each terminal is transmitted to the information management server 32a to record information and determine a health condition on the information management server 32a side. The determination result or the recorded information can be transmitted to the user terminal 50a or the external terminal 50b, and can be confirmed by each terminal. Thus, the system structure which gives the function of the software 1a on an external server can also be adopted.

### (Third System Configuration)

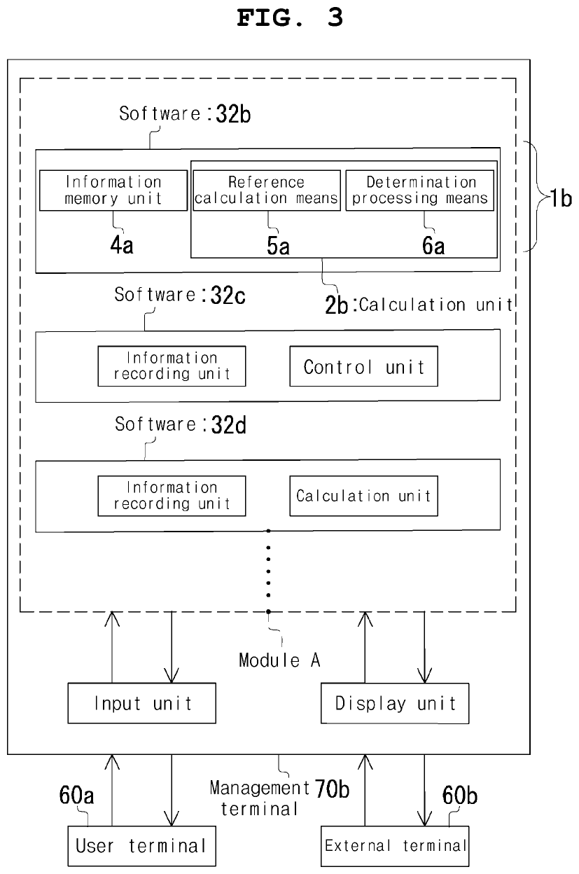

In FIG. 3, a configuration of a management terminal 70b including a module A having a plurality of software 32c and 32d and the like in addition to the function of software 32b to which the present disclosure is applied, as a third system configuration is shown. The software 32b to which the present disclosure is applied constitutes the module A together with other software for enabling the management terminal 70b to perform various functions different from itself. In other words, it is possible to mount the software 32b on the module A of the management terminal 70b in which the plurality of software 32c and 32d, or the like has been introduced in advance, and make it function. For example, the software to which the present disclosure is applied can also be mounted in the module included in a management terminal of a medical system such as an electronic chart.

In this third system configuration, vital information is input to the management terminal 70b to perform determination of the health condition, and the result information can be confirmed on the management terminal 70b. In addition, the user terminal 60a or the external terminal 60b are connected to the management terminal 70b so that the vital information is input from the user terminal 60a or the external terminal 60b and transmitted to the management terminal 70b, the management terminal 70b performs determination of the health condition, and the result information may be received and confirmed by the user terminal 60a or the external terminal 60b. As such, the software to which the present disclosure is applied can also adopt a configuration that functions as part of a module composed of a plurality of software.

As described above, the configuration on the system of the software (or health condition determination device) to which the present disclosure is applied has a plurality of variations. In addition, although the above description has been mainly focused on three examples, the configuration of the software (or health condition determination device) to which the present disclosure is applied is not limited thereto. For example, an information recording unit may be provided in the user terminal, and the reference calculation means and the determination processing means may be given to an external server to divide the location of necessary functions into the terminal and the server. That is, if the vital information of the subject is recorded, the determination reference reflecting the variation in the individual is configured, and the determination of the health condition is possible, various configurations can be adopted.

Detailed configuration will be described below using the usage aspect of the tablet terminal 3 shown in FIG. 1.

### [2. Information Recording Unit]

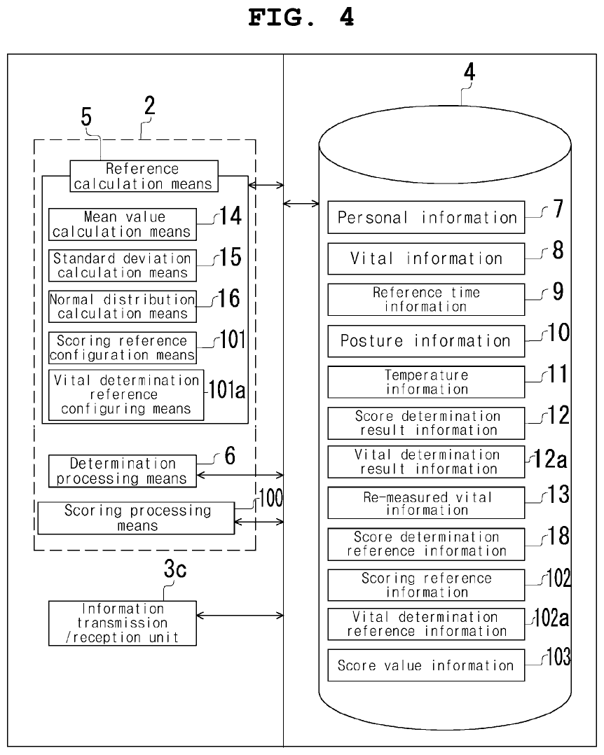

As shown in FIG. 4, various information is recorded in the information recording unit 4.

The information recording unit 4 is a part that stores the vital information including the personal information of the subject and the value of the vital signs measured by various vital meters, and the evaluation result of the consciousness level obtained by observation of the subject's caregiver and the like with information of measurement date and time or acquisition date and time. The various information recorded in the information recording unit 4 can be input or corrected through the input unit 3a, the information transmission/reception unit 3c, and the information input unit 23 (not shown) included in the tablet terminal 3. Moreover, content of the various information recorded in the information recording unit 4 can be confirmed through the display screen 3b and the information transmission/reception unit 3c which are included in the tablet terminal 3.

The information recording unit 4 stores the personal information 7 of the subject, the measurement value of the vital signs measured by each vital meter, the evaluation result of the consciousness level obtained by observation for the subject, and vital information 8 including the information of the measurement date and time or acquisition date and time. In addition, the personal information 7 and the vital information 8 are configured to be recordable in association with identification information that can identify individual subjects. As a result, the plurality of subjects can be identified, and the plurality of subjects can use one health condition determination device 1.

The vital information 8 includes measurement values of body temperature, pulse, diastolic blood pressure, systolic blood pressure, pulse pressure, and respiratory rate. In addition, the vital information 8 includes a measurement value of oxygen saturation. Furthermore, the vital information includes the evaluation result of the above-mentioned consciousness level.

In addition, the measurement date and time or acquisition date and time included in the vital information 8 are the date and time when the subject performed the vital measurement or the confirmation of the consciousness level. For example, the time when the subject performed the vital measurement by himself or the time when the caregiver observed the subject is confirmed and then input. In addition, when the vital meter is a wearable measuring device that can be worn on the subject's body, the measurement date and time or acquisition date and time may be the acquisition date and time of continuously acquired vital signs.

Here, the type of vital information is not necessarily limited to the measurement values of body temperature, pulse, systolic blood pressure, diastolic blood pressure, pulse pressure, respiratory rate, the measurement value of oxygen saturation, and evaluation result of consciousness level, and may include other vital signs (e.g., amount of urine) to be used for determination.

In addition, the vital meter for measuring the vital information is not particularly limited, and it is sufficient if each vital sign can be measured. For example, the vital signs may be measured using a vital meter for home. More specifically, it is not necessary to use a vital meter if vital information can be obtained. For example, it is also possible to measure the pulse rate or the respiratory rate for 1 minute while measuring the time with a clock, and use it as vital information. However, from the viewpoint of accurately determining the variations in the individual of vital information, the vital information is preferably obtained by the same technique. In the daily measurement, the type of vital meter is frequently changed, or the measurement by the vital meter and the measurement without the vital meter are mixed, so that differences arise depending on how the vital sign is measured. Therefore, it is desirable to obtain the vital information by using the same technique or the same vital meter as much as possible.

In addition, the vital information 8 is configured to be capable of recording vital information 8 broadly every second. In addition, the vital information 8 may be configured to be recorded at different time intervals, for example, every minute, every hour, etc.

Further, the vital information 8 may employ a configuration in which measurement values measured at irregular times are recorded instead of being measured at regular intervals. In addition, in the case of this irregular measurement, it is preferable to use a structure which records multiple pieces of vital information 8 during a certain period of time, for example, multiple pieces of vital information 8 are acquired for one minute, multiple pieces of vital information 8 are acquired for 30 minutes, multiple pieces of vital information 8 are acquired for 1 hour, multiple pieces of vital information 8 are acquired for several hours, multiple pieces of vital information 8 are acquired for one day, multiple pieces of vital information 8 are acquired for several days, multiple pieces of vital information 8 are acquired for one week, multiple pieces of vital information 8 are acquired for several weeks, or multiple pieces of vital information 8 are acquired for one month.

Furthermore, the vital information 8 may be recorded as the vital information 8 for multiple pieces of measurement data by randomly extracting multiple pieces of data from the accumulated vital information regardless of a regular interval or irregular interval.

As described above, the vital information 8 is configured to be capable of recording multiple pieces of measurement data regardless of the length of time and the regularity of the measurement interval.

In addition, the vital information 8 is configured to be capable of recording vital information measured, for example, twice a day in the morning and evening time zones.

In addition, the information recording unit 4 is capable of recording the reference time information 9 which is information of the time at which the subject performs the vital measurement. In the reference time information 9, for example, a reference time for measuring the vital information of the subject is recorded, such as 8:30 in the morning and 18:00 in the evening. The reference time information 9 can be freely configured and corrected by the subject.

In the information recording unit 4, posture information 10, which is information of a correct posture when measuring the value of each vital sign, is recorded. The posture information 10 is as follows, for example.

### (1) Body Temperature

For example, when measuring body temperature with a thermometer that measures a body temperature under the armpits, body temperature is information indicating a posture of “whether the thermometer is located at the center of armpits”, “whether armpits and thermometer are in close contact”, and “the same posture is taken each time” or the like.

### (2) Pulse

For example, when measuring a pulse by applying an electronic pulse meter or fingers on the wrist, a pulse is information indicating a posture of “whether the subject is in a stable state”, “whether the subject has a relaxed posture”, and “whether the same posture is taken every time” or the like.

### (3) Systolic Blood Pressure and Diastolic Blood Pressure

For example, when measuring in an oscillometric method, which measures the vibration of blood vessels, systolic blood pressure and diastolic blood pressure is information indicating the posture of, for example, “whether the subject is in stable state”, “whether a wound arm or wrist is at the height of the heart”, “whether the same posture is taken every time” or the like.

Here, the vital information 8 is not necessarily configured to be capable of recording vital information measured twice a day in the morning and evening time zone, and may be, for example, measured once a day. As will be described later, if a certain number of data is used in the calculation of the determination reference by the reference calculation unit, the vital mean value used for the calculation of this determination reference, and the calculating processing for the vital standard deviation, is recorded, the number of recording of vital information for one day is not limited. In addition, the vital information need not always be recorded every day, and there may be days when the vital information is not recorded. Here, from the viewpoint of properly determining the intra-individual variation of the same individual, the aspect which records vital information every 1 second is generally good, and it is preferable to record the vital information once to 24 times a day. Furthermore, even in manual vital measurement, it is easy to record information, and it is possible to check the change in the value of vital signs on the same day, and it is easy to compare with other days, and thus it is further preferable to record vital information which is measured twice a day in the morning and evening time zone.

In addition, the reference time information 9 does not necessarily need to be recorded in the information recording unit 4. However, as will be described later, by recording the reference time information 9, it is possible to make determination by excluding the vital information of the subject measured at the time that deviates significantly from the time recorded in the reference time information 9 from the calculation basis such as the vital mean value, thereby increasing the accuracy of determination. Therefore, it is preferable that the reference time information 9 is recorded in the information recording unit 4.

In addition, the posture information 10 does not necessarily need to be recorded in the information recording unit 4. However, as will be described later, by recording the posture information 10, when it is determined that the vital information of the subject is an abnormal value, attention about posture at the time of vital measurement or vital re-measurement can be urged by displaying “did you perform the measurement with the correct posture” while displaying, on the display screen 3b of the tablet terminal 3, the posture information 10 of the vital sign that is the basis of the determination. Thereby, the accuracy of vital measurement and the reliability of determination can be improved. Therefore, it is preferable that the posture information 10 is recorded in the information recording unit 4.

In addition, the content of the measurement method or the posture information 10 of each vital sign is not limited to the above descriptions, and the content of the vital measurement method or the suitable posture information 10 may be suitably changed.

The information recording unit 4 is capable of recording the temperature information 11 of a place where the vital information is measured. The temperature information 11 is recorded in association with the recording at the time of measurement of the vital information 8. The temperature information 11 adopts the information which a subject confirms and inputs as the temperature of a measurement place, for example.

Here, it is not always necessary to record the temperature information 11 of the place where the vital information is measured in the information recording unit 4. However, if the temperature information on the determination day and the temperature information of the vital measurement performed the day before the determination day are compared and the displacement amount of the two temperature information exceeds the configured range, the vital information on the determination day may be excluded from the calculation basis of the mean value or vital standard deviation obtained thereafter. As a result, the influence of the temperature on the variation of vital information can be reduced and thus the accuracy of the determination may be increased. Accordingly, it is preferable that the temperature information 11 of the place where the vital measurement is performed can be recorded in the information recording unit 4.

As shown in FIG. 4, in the information recording unit 4, vital determination reference information 102a serving as a reference when the determination processing means 6 determines whether the value of the input vital sign is an abnormal value is recorded.

The vital determination reference information 102a is capable of adding or modifying information via the input unit 3a of the tablet terminal 3, the information transmission/reception unit 3c, and the information input unit 23 of the calculation unit 2.

In the information recording unit 4, the vital determination result information 12a, which is information of the determination result when the determination processing means 6 determines whether the value is an abnormal value with respect to the value of the vital sign, is recorded. The content can be confirmed through the display screen 3b of the tablet terminal 3.

In addition, in the information recording unit 4, the re-measured vital information 13, which is the vital information when the measurement and the like are re-performed in relation to the measurement and acquisition of the vital information and the date at the time of the measurement, can be recorded as the vital information 8. The re-measured vital information 13 may be, for example, vital information re-measured to confirm the accuracy of the vital information when the determination processing means 6 determines that the score value obtained for the vital information is an abnormal value.

In the present disclosure, the re-measured vital information 13 can be employed as the calculation basis as a basis for calculating the vital determination reference information and the scoring reference information.

In addition, when each vital information is displayed on the display screen 3b of the tablet terminal 3, characters representing vital information of three patterns can be displayed in different colors, with respect to normal vital information recorded without re-measurement, vital information targeted for re-measurement, and vital information after re-measurement.

As shown in FIG. 4, the scoring reference information 102 serving as a reference when scoring each vital information to be input by the scoring processing means 100 is recorded in the information recording unit 4. In addition, score value information 103 which is information on the numerical value obtained as a result of scoring based on the scoring reference information 102 is recorded in the information recording unit 4.

In addition, score determination reference information 18 serving as a reference when the determination processing means 6 determines whether the score value information obtained from the content of the input vital information is an abnormal value is recorded in the information recording unit 4.

The scoring reference information 102 and the score determination reference information 18 to be described later can add or modify information via the input unit 3a of the tablet terminal 3, the information transmission/reception unit 3c, and the information input means 23 of the calculation unit 2. Further, the contents of each scoring reference information 102 may be confirmed via the display screen 3b of the tablet terminal 3. In addition, the detailed contents of each reference in the scoring reference configuring means 101 will be described later.

In the information recording unit 4, score determination result information 12, which is information of a determination result of determining whether the score value information 103 is an abnormal value by the determination processing means 6, is recorded. The content can be confirmed through the display screen 3b of the tablet terminal 3. In addition, the score determination result information 12 may indicate the determination result by color classification according to the score as well as the display of abnormality or normality. For example, display by color classification, such as red for 3 or more points, yellow for 2 points, and no color for 1 or less points, can be possible.

Further, the score determination result information 12 may be a result of determining not only the score value information 103 for an individual but also the total score obtained by adding multiple pieces of (for example, all or a part) score value information 103. In this case, the determination result may be indicated by the determination of abnormality or normality or color classification according to the score for the total score obtained by adding multiple pieces of score value information 103.

For example, the score determination result information 12 on a certain determination day can perform abnormality determination based on the scoring of whether it is abnormal with respect to the total score of the score value information of the scoring process based on the value of the vital sign measured on that day.

Here, it is not necessary to make it possible to record the score determination result information 12 and the vital determination result information 12a in the information recording unit 4. However, it is preferable to enable recording of the score determination result information 12 and the vital determination result information 12a in the information recording unit 4, in that the result of the determination of past vital information can be confirmed or used as reference information for improving the accuracy of the determination and can be used as a reference to the diagnosis result of the doctor or even as a linkage to the medical system.

In addition, it is not necessary to enable the re-measured vital information 13 to be recorded in the information recording unit 4. However, it is preferable that the re-measured vital information 13 be recordable in the information recording unit 4 in that it is possible to verify whether the vital measurement was accurate using the re-measured vital information 13, and the accuracy of the determination is easily increased by forbidding the value of the vital sign with poor measurement method and poor measurement accuracy from being included as the basis for the determination reference.

### [3. Reference Calculation Means]

A reference calculation means 5 will now be described. The reference calculation means 5 is one of the functions that the software to which the present disclosure is applied executes on the calculation unit 2, and calculates the numerical range for the vital determination severing as the vital determination reference information 102a for determining whether the vital information (vital information to be input) recorded in the information recording unit 4 is an abnormal value with respect to the value of the vital sign or performs a calculating processing of the vital mean value and the vital standard deviation used for the calculation of the numerical range for vital determination serving as the vital determination reference information 102a. In the health condition determination device 1, the reference calculation means 5 calculates the numerical range for the vital determination severing as the vital determination reference information 102a with respect to the measurement values of body temperature, pulse, systolic blood pressure, diastolic blood pressure, pulse pressure, and respiratory rate, and this numerical range becomes a reference for determining whether it is an abnormal value for the value of the vital signs.

Further, the reference calculation means 5 calculates the numerical range which becomes scoring reference information 102 for calculating score value information 103 with respect to the vital information recorded (input vital information) in the information recording unit 4 or perform a calculating processing for the vital mean value and the vital standard deviation used for calculation of the numerical range for the scoring reference information 102. In the health condition determination device 1, the reference calculation means 5 calculates the numerical range which becomes scoring reference information 102 with respect to the measurement values of body temperature, pulse, systolic blood pressure, diastolic blood pressure, pulse pressure, and respiratory rate, and this numerical range becomes a reference for scoring.

Various types of information calculated or recorded by causing the calculation unit 2 to function as the reference calculation unit 5 can be added or modified via the input unit 3a of the tablet terminal 3, the information transmission/reception unit 3c, and the information input unit 23 of the calculation unit 2. Further, the contents of the various information calculated or recorded by causing the calculation unit 2 to function as the reference calculation means 5 can be confirmed through the display screen 3b of the tablet terminal 3.

FIG. 4 shows a function by which the software to which the present disclosure is applied is executed in the calculation unit 2. The calculation unit 2 functions as a mean calculation means 14, a standard deviation calculation means 15, a normal distribution calculation means 16, a scoring reference configuring means 101, and a vital determination reference configuring means 101a which constitute the reference calculation unit 5.

In addition, the mean calculation means 14 and the standard deviation calculation means 15 calculate the “standard deviation of the vital information” in the distribution of the total vital information under the same condition as the “mean value of the vital information” under the same condition from the recorded information under a predetermined condition, respectively, based on the vital information 8 (measurement values of body temperature, pulse, systolic blood pressure, diastolic blood pressure, pulse pressure, and respiratory rate) recorded in the information recording unit 4 and re-measured vital information 13 thereof. Further, in the following description, the mean value of the vital information is referred to as “the vital information mean value”, and the standard deviation of the vital information is referred to as “the vital information standard deviation”, except for the case the name of the mean value or the standard deviation of the type for performing a special calculation is indicated. In addition, predetermined conditions are mentioned later.

In addition, for the vital information 8 recorded in the information recording unit 4, the mean calculation means 14 and the standard deviation calculation means 15 can perform both a pattern of calculating (1) the vital information mean value and the vital information standard deviation, including the value of the vital sign when the value of the vital sign at the input determination time point is determined to be an abnormal value, based on the vital determination reference information 102a, and a pattern of calculating (2) the vital information mean value and the vital information standard deviation, excluding the value of the vital sign when the value of the vital sign at the input determination time point is determined to be an abnormal value, based on the vital determination reference information 102a.

In addition, for the vital information 8 recorded in the information recording unit 4, the mean calculation means 14 and the standard deviation calculation means 15 can use two distinguished patterns including a pattern of calculating (1) the vital information mean value and the vital information standard deviation, including the value of the vital sign when the score value information 103 is determined to be an abnormal value, based on the score determination reference information 18, and a pattern of calculating (2) the vital information mean value and the vital information standard deviation, excluding the value of the vital sign when the score value information 103 is determined to be an abnormal value, based on the score determination reference information 18.

In this way, by calculating the vital mean value or the vital standard deviation to include not only the value of the vital sign that is determined to be normal but also the value of the vital sign that is determined to be abnormal, the mean value or the standard deviation reflecting the variation in the individual of the subject can be obtained. Further, by using such a mean value and a standard deviation, it is possible to establish a reference that reflects the variation in the individual of the subject when configuring the scoring reference information 102 or the vital determination reference information 102a.

In addition, the accuracy of determination can be increased by having a pattern in which the vital mean value or vital standard deviation is calculated by excluding the value of the vital sign that is the basis determined as abnormality, for example, by forbidding the vital information which is unstable under special circumstances from being included in the calculation basis of the determination reference. The vital information that is not stable under special circumstances as used herein means, for example, a value of vital signs measured at the time of medical intervention for a subject, that is, immediately after the subject is hospitalized by a doctor's diagnosis (instruction). The value of vital signs measured under such circumstances can easily have an unstable value in view of intra-individual variation of the subject's vital signs, and thus this value is excluded from the calculation basis of the determination reference.

In addition, the mean value calculation means 14 and the standard deviation calculation means 15, for the vital information 8 recorded in the information recording unit 4, can configure a pattern of calculating the mean value and the standard deviation of vital information, except for the values of vital signs measured from the subject in a predetermined state. Accordingly, the value of the vital sign measured from the subject in the predetermined state is excluded from the calculation basis of the vital determination reference information 102a and the scoring reference information 102. This predetermined state is a special state in which the vital signs of the subject are not stable. For example, the value of body temperature measured in a state where the subject's body temperature is not stable (does not show an original tendency to change) by taking the antipyretic agent is excluded from the calculation basis of the determination reference. Thereby, the accuracy of the determination of abnormality on the vital signs for a short period of time can be increased.

The “predetermined condition” employed in the calculation of the mean value calculation means 14 and the standard deviation calculation means 15 employs a method in which n pieces of vital information (body temperature, pulse, systolic blood pressure, diastolic blood pressure, pulse pressure, and respiratory rate) is used generally starting from the determination time point. The vital information for this period employs two distinguished patterns including (1) a pattern using the n pieces of past vital information 8 and re-measured vital information 13 without including measurement data at the determination time point (targeted for the determination), and (2) a pattern using the n pieces of past vital information 8 and re-measured vital information 13, including the measurement data at the determination time point (targeted for the determination).

Here, as for n pieces of vital information, (1) measurement of vital signs once a day is performed and vital determination reference information 102a and scoring reference information 102 are generated from vital information for 4 days or more (n=4 or more) to perform abnormality determination for vital signs and an abnormality determination based on scoring. In addition, as for n pieces of vital information, (2) measurement of vital signs twice or more per day is performed, and vital determination reference information 102a and scoring reference information 102 are generated from vital information for 2 days or more (n=2 or more) to perform abnormality determination for vital signs and an abnormality determination based on scoring.

In addition, n pieces of vital information may increase the date of acquiring vital information to generate vital determination reference information 102a and scoring reference information 102. For example, by increasing the number of data by 1 day after the 4 days, for example, by increasing days to acquire vital information, such as 10 days, 14 days, 30 days, 60 days, 90 days, 120 days, 365 days, the vital determination reference information 102a and the scoring reference information 102 may be generated based on the vital information for the days.

In addition, as described above, the configuration for n pieces is vital information data measured broadly every second, and in addition, a different length of time may be employed together with vital information data measured every minute, every several minutes, every hour, every day, and every month. It is also possible to extract multiple pieces of irregularly acquired data. At this time, the configuration may simply include a method of extracting multiple pieces such that the acquired order is checked back. Also, the configuration may also include a method of configuring a certain extraction condition for irregularly acquired data to extract multiple pieces. The extraction condition may also be, for example, a condition for extracting multiple pieces within a predetermined range of 1 hour, or a condition in which the interval of the acquisition time between vital information satisfies a certain condition (the interval is at least 5 minutes or more or the interval is within 1 hour). Furthermore, the configuration may also include a method of selecting and extracting multiple pieces of vital information 8 at random with respect to the vital information 8 regularly measured at regular intervals. The extraction conditions for multiple pieces may be appropriately configured as needed.

In addition, as described above, the vital information 8 is configured to be capable of being recorded broadly every second. The vital information 8 may be configured to be recorded at different time intervals, for example, every minute, every hour. Furthermore, the vital information obtained by measuring irregularly several times a day may be configured to be recorded. When the calculation unit 2 functions as the mean value calculation means 14, the standard deviation calculation means 15 to calculate the vital mean value and the vital standard deviation, the vital mean value and the vital standard deviation may be calculated appropriately, under the configuration conditions.

In addition, at the determination time point of the value of the score value information 103 or the value of the vital sign based on the input vital information of a subject, the mean value calculation means 14 and the standard deviation calculation means 15 refer to the vital information 8 and the re-measured vital information 13 recorded before every determination time point, and calculate the mean value of the vital information and the standard deviation of the vital information. Thereby, the reference used by the determination processing means 6 (or the score processing means 100) is changed for each determination time point, and thus it becomes easy to reflect the intra-individual variation of the subject's vital information in the determination of whether the value of the vital sign is an abnormal value and in the determination of whether the score value information 103 based on the vital information is an abnormal value.

In addition, there may be a configuration using a larger number of pieces of vital information 8, such as 10, 14, 30 or 90 or more. By increasing the number of pieces of vital information 8, normality of the vital information 8 may be easily obtained. In addition, at least 4 pieces of data is preferable as the minimum number for identifying the variation in the individual of the subject.

In addition, the “predetermined condition” employed at the time of calculation of the mean calculation means 14 and the standard deviation calculation means 15 does not necessarily need to be vital information measured on consecutive days (numbers). For example, in a case where there is a day (timing) when the vital information is not recorded when there is a day (timing) when the subject does not perform the vital measurement, the number of days (pieces) of a predetermined condition may be “total 4 days (4 pieces)”.

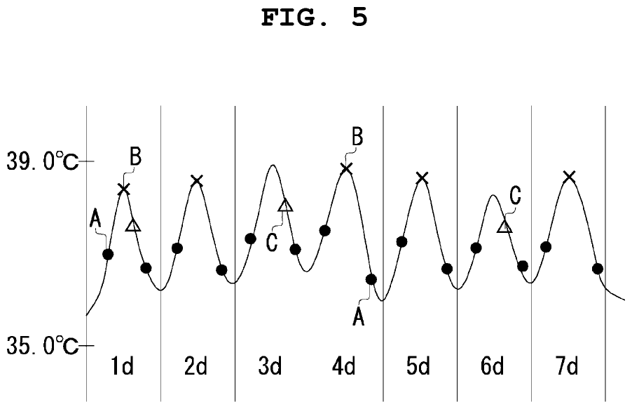

For example, as indicated by the symbol A (black circle) in FIG. 5, the vital information is recorded twice in the morning and afternoon every day, and all the information is used for the calculation of the mean value calculation means 14 and the standard deviation calculation means 15.

At this time, in the present disclosure, if the data number of the configured number of vital information is provided, it does not necessarily need to be the vital information continuously acquired every second, every minute, every hour, every day. Like the vital information shown by the symbol B (the figure of X) or the symbol C (white triangle) of FIG. 5, there may be an aspect in which the date (timing) of acquiring vital information is discontinuous and is acquired once in several days (several times). Furthermore, there may be an aspect in which information is partially extracted based on the configured conditions in the state where continuous recording of vital information exists. The configured conditions are contents such as extracting only vital information on every Monday, only vital information acquired in the morning, extracting on only a specified date, and the like.

In addition, the normal distribution calculation means 16 is a part for calculating the normal distribution from the mean value and the standard deviation of the vital information under predetermined conditions. A normal distribution at each determination time point of the subject can be calculated, and a normal distribution curve obtained by graphing the established density function is created for the calculated normal distribution, and this normal distribution curve is displayed on the display screen 3b of the tablet terminal 3.

In addition, the vital determination reference configuring means 101a interworks with the mean value calculation means 14 and the standard deviation calculation means 15 to generate the vital determination reference information 102a used for determining by the determination processing means 6, based on the vital mean value and the vital standard deviation calculated from each calculation unit. The generated vital determination reference information 102a is recorded in the information recording unit 4.

More specifically, the vital determination reference configuring means 101a interworks with the mean value calculation means 14 and the standard deviation calculation means 15 to generate the vital determination reference information 102a used for determining the value of the vital signs, in reference to the measurement values of body temperature, pulse, systolic blood pressure, diastolic blood pressure, and pulse pressure, and respiratory rate measured from the subject, based on the vital mean value and the vital standard deviation calculated from the respective calculation means.

In addition, the scoring reference configuring means 101 interworks with the mean value calculation means 14 and the standard deviation calculation means 15 to generate the scoring reference information 102 used for scoring by the scoring processing means 100, based on the vital mean value, the vital standard deviation, and the mode calculated from each calculation unit. The generated scoring reference information 102 is recorded in the information recording unit 4.

More specifically, the scoring reference configuring means 101 interworks with the mean value calculation means 14 and the standard deviation calculation means 15 to generate the scoring reference information 102 used for scoring, in reference to the measurement values of body temperature, pulse, systolic blood pressure, diastolic blood pressure, pulse pressure, and respiratory rate measured from the subject, based on the vital mean value and the vital standard deviation calculated from the respective calculation means.

In addition, the scoring reference information 102 includes not only the calculation result of each calculation means but also information of a predetermined fixed numerical range used when scoring the measurement value of oxygen saturation, or a predetermined observation state capable of distinguishing the degree of consciousness level.

More specifically, for the measurement value of oxygen saturation measured from the subject, the predetermined numerical range may be input from the input unit 3a of the tablet terminal 3, and may be configured as the scoring reference information 102. The configured scoring reference information 102 is recorded in the information recording unit 4.

In addition, for the evaluation result of the consciousness level acquired from the subject, the content of the predetermined observation state which can distinguish the degree of consciousness level may be input and then be configured as the scoring reference information 102. The configured scoring reference information 102 is recorded in the information recording unit 4. In addition, the details of the calculation of the vital mean value, the vital standard deviation, the mode, and the scoring reference information 102, or the configuring of the scoring reference information 102 composed of a plurality of items will be described later.

### [4. Scoring Processing Means]

The scoring processing means 100 will now be described. The scoring processing means 100 is one of the functions that the software to which the present disclosure is applied, executed in the calculation unit 2, and calculates score value information 103 (information of score) according to the contents of the vital information, for the vital information at the determination time point input through the input unit 3a of the tablet terminal 3, based on the processing information of the mean value calculation means 14 and the standard deviation calculation means 15 or the scoring reference information 102 including the preconfigured reference.

The score value information 103 calculated by the scoring processing means 100 is recorded in the information recording unit 4, as described above. At this time, the score value information 103 is recorded in association with the identification information which can identify an individual and the information used as the calculation reference of a score value. The scoring processing means 100 is configured to calculate the score value information 103 in association with the information recording unit 4 and the reference calculation means 5.

In addition, the content of the score value information 103 can be confirmed through the display screen 3b of the tablet terminal 3. In addition, the score value information 103 may be confirmed on these screens and the like by sending score determination result information 12 to an external server or an external terminal through the information transmission/reception unit 3c of the tablet terminal 3 as well as the display screen 3b of the tablet terminal 3. The content of the score value information 103 may be displayed as an individual numerical value or a total score of a plurality of score values at the determination time point of the same individual.

### [5. Determination Processing Means]

The determination processing means 6 will now be described. The determination processing means 6 is one of the functions that the software to which the present disclosure is applied executes in the calculation unit 2, and determines whether the value of the vital sign is an abnormal value with respect to the value of the vital signs at the input determination time point, based on the vital determination reference information 102a.

In addition, the determination processing means 6 determines whether the scoring value information 103 is an abnormal value, for the scoring value information 103 obtained by scoring vital information at the determination time point, which is input through the input unit 3a of tablet terminal 3, based on the score determination reference information 18.

As described above, the score determination result information 12 and the vital determination result information 12a, which are the determination results determined by the determination processing means 6, are recorded in the information recording unit 4. In addition, the contents of the score determination result information 12 and the vital determination result information 12a may be confirmed through the display screen 3b of the tablet terminal 3. In addition, the score determination result information 12 and the vital determination result information 12a may be transmitted to an external server or an external terminal through the information transmission/reception unit 3c of the tablet terminal 3 as well as the display screen 3b of the tablet terminal 3, and confirmed on these screens and the like.

In addition, the score determination result information 12 and the vital determination result information 12a may be not only displayed on the display screen 3b of the tablet terminal 3, but may also be notified to the subject by a notification sound or a mail message which notifies that the score determination result information 12 and the vital determination result information 12a have come out. When notifying of the score determination result information 12 and the vital determination result information 12a by the notification sound, it is also possible, for example, to change the type of notification sound in the case of an abnormal value and in the case where it is not.

### [6. Re-measurement of Vital Signs]

When the vital information is determined as an abnormal value, the message “Do you want to perform the measurement again?” may be displayed on the display screen 3b of the tablet terminal 3 to promptly request the re-measurement of the vital measurement. In addition, as described above, the posture information 10 recorded in the information recording unit 4 is displayed to display a message of “Did you perform vital measurement in the correct posture?” Furthermore, it is possible to display the message “Is the vital measurement measured at a certain measurement time?”

In this way, vital information is re-measured by urging attention to the subject who has input the vital information and performing a reply that the subject himself/herself performs re-measurement of the vital information through the input unit 3a of the tablet terminal 3, and the information result may be recorded in the information recording unit 4, which becomes the re-measured vital information 13.

The re-measured vital information 13 can be used as a calculation basis for the subsequent vital mean value, vital standard deviation, vital determination reference information 102a, and scoring reference information 102. When each vital information is displayed on the display screen 3b of the tablet terminal 3, characters representing vital information of three patterns can be displayed in different colors, with respect to normal vital information recorded without re-measurement, vital information targeted for re-measurement, and vital information after re-measurement.

Moreover, as another determination method by the determination processing means 6, when a vital mean value matches a predetermined condition, a method of determining as “there is a possibility of going to abnormality” will be described. Here, the vital information recorded in the information recording unit 4 is used to compare the vital mean value for the last 7 days with the vital mean value for the last 30 days, and when the difference between the two vital mean values exceeds a predetermined range, the determination processing means 6 determines that “there is a risk of going to an abnormality.”

Here, a predetermined displacement in the difference between the two mean values may be configured as a value of 0.5 σ or more based on, for example, the vital standard deviation σ of the determination date. The vital mean values for the last 7 days on the determination date and for the last 30 days on the determination date is expected to be the same value as usual, even if there is intra-individual variation in the subject's vital information. However, if the difference between the two vital mean values is 0.5σ or more, there is a large variation in the vital mean value, and with this phenomenon, it cannot be determined that the subject has an “abnormal value”, but determined that “There is a risk of going to an abnormality.”Therefore, the determination can be used as an index that the condition may deteriorate in the future.

In this way, by comparing the difference between the two vital mean values in the range of a certain period of time, the determination processing means 6 performs a determination suggesting deterioration of the condition that “there is a risk of going to an abnormality” to alert the subject or to link the subject to preventive medicine. In addition, the number of days such as the last 7 days and the last 30 days is not necessarily limited thereto. In addition, the vital information of a determination date includes both an aspect which the calculation basis of a vital mean value is included and an aspect in which the calculation basis of a vital mean value is not included.

Next, a description will be given of specific contents of a device and an input screen to be used when executing the software to which the present disclosure is applied.

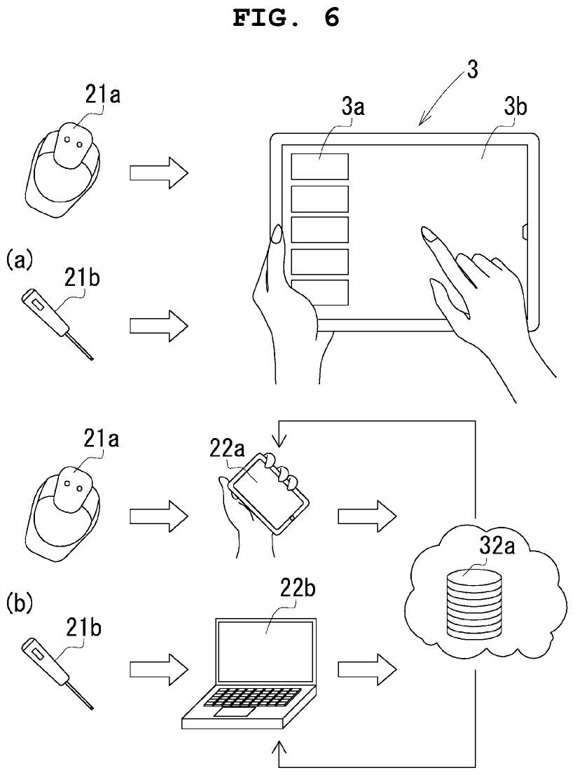

For example, as shown in FIG. 6A, the vital information is acquired by a wearable vital meter 21a, a thermometer 21b, or the like, and the measurement values measured thereby are input via the screen displayed on the display screen 3b of the tablet terminal 3 with the information of the measured time. On the display screen 3b, an input unit 3a in the form of a touch panel is displayed, and vital information is input thereto. If the tablet terminal 3 to which the software to which the present disclosure is applied is introduced (first system configuration), the terminal itself can display the recording of information, the determination of the condition of health and the determination result.

In addition, as shown in FIG. 6B, it is also possible to access the information management server 32a, which is the external server described in the above-described second system configuration, from a smart phone terminal 22a or a personal computer terminal 22b (hereinafter, referred to as a “PC terminal 22b”) to input vital information through the smart phone terminal 22a or the PC terminal 22b. Based on the vital information transmitted from each terminal, the determination of the condition of health is made in the information management server 32a, and the result information is transmitted to each terminal, and the result information is displayed on the screen of each terminal.

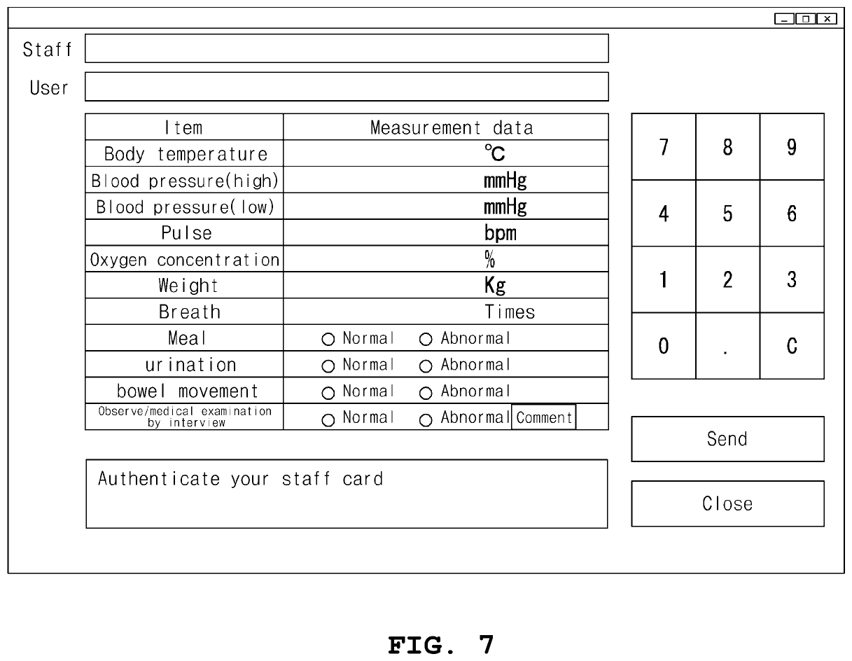

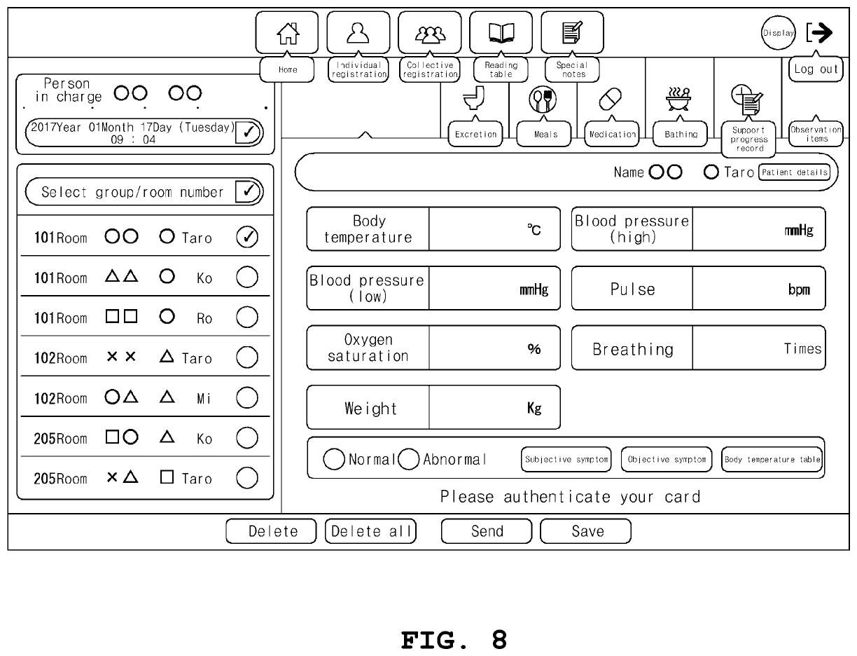

Moreover, the screen shown in FIG. 7 and FIG. 8 is shown as an input screen of the tablet terminal 3, the smart phone terminal 22a, and the PC terminal 22b. FIGS. 7 and 8 are examples of input screens used when a patient in a hospital or a resident, such as a nursing facility, is to be determined as a health object. In FIG. 7, the input item of one person and the ten-key area which displays numbers are displayed. The name display column of the subject and the staff in charge, and input fields of measurement data in body temperature, blood pressure (up and down), pulse, oxygen concentration, weight, and respiratory rate are provided. As for the value of each vital sign, input to the ten-key area can be achieved by a touch panel or a cursor operation on the screen.

In addition, in the screen display of FIG. 7, items of diet, urination, bowel movement, and observation/check-up are provided, and in addition to the value of the vital sign, a plurality of items for confirming the health condition of the subject are also provided. By recording the plurality of items identifying health conditions, it is possible to make a record of the subject's daily health conditions and become information usable at the time of calculation of the determination reference of vital information mentioned later. The input information is recorded in the vital information inside the device by touching or clicking the transmit button or is transmitted to the information management server 32a outside.

In the input screen illustrated in FIG. 8, input fields of measurement data of a plurality of vital signs and selection items of normal or abnormal conditions determined by the subject on their own are provided on the right side of the screen. In addition, subjective sign, objective sign, and a thermal table can be selected to input additional conditions of information or to identify changes in the vital signs of the subject.

In addition, in the screen of FIG. 8, the names of the plurality of subjects are displayed, and the screen of the selected subject can be displayed by selecting the name field. Moreover, the information of the time at the time of input of the value of a vital sign is input simultaneously. Furthermore, in addition to the input screen of the value of the vital sign, it is also possible to record or display information on items of information registration, and information on care items such as excretion and meals to be provided.

As described above, the input screen when using the software of the present disclosure can be used for the patient of a hospital, a care facility, or the like, so that input or information can be displayed together with related items. In addition, the display of the input screen is not limited to the contents associated with the caregiver and the like, and is, for example, a screen configuration that combines input of the value of each vital sign and management of information such as record and weight as application software for health care. That is, it can also be set as the aspect in which a healthy subject uses for daily health care.

Next, a specific determination method based on vital information will be described.

### [7. About Calculation of Vital Mean Value, etc., Abnormality Determination of Vital Signs, Abnormality Determination Based on Scoring]

### [7-1. About Measurement of Body Temperature, Pulse, Systolic Blood Pressure, Diastolic Blood Pressure, Pulse Pressure]

The vital mean value and the vital standard deviation are calculated by the calculation unit 2 functioning as the mean value calculation means 14 and the standard deviation calculation means 15 of the reference calculation means 5, based on the vital information 8 and the re-measured vital information 13 recorded in the information recording unit 4. In addition, the scoring reference information 102 and the vital determination reference information 102a for the measurement values of the body temperature, pulse, systolic blood pressure, diastolic blood pressure, pulse pressure, and respiratory rate are configured based on the vital mean value and the vital standard deviation.

In addition, the configuring of the determination reference of the plurality of patterns and the contents of the determination, employed in the present disclosure are changed by the determination reference configuring means 17 to enable the selection of the determination method to be used or the selection of the method of combining the plurality of patterns.

As a method for configuring the vital mean value, the vital standard deviation, the scoring reference information 102, and the vital determination reference information 102a based on the vital mean value and the vital standard deviation, there may be a method of using the vital information 8 and the re-measured vital information 13 recorded in the information recording unit 4 to calculate the vital mean value, and the like. In this method, the standard deviation based on the vital mean value and the distribution of the vital information is calculated using the following equations (3) and (4) in the mean value calculation means 14 and the standard deviation calculation means 15:

μ=(1/N)×ΣSi Equation (3); and

σ=√(1/N)×Σ(Si-μ)2) Equation (4).

Here, μ is the mean value of the vital information, Si is the measurement value of each vital information, N is the number of data of the entire vital information, and σ is the standard deviation. ρSi represents the sum of measurement values of all vital information. In addition, the measurement value of each vital information is the value of the vital information acquired on the predetermined extraction condition configured as mentioned above. In addition, the content of all vital information here may be that extracted as a part of the information recorded in the information recording unit 4 as mentioned above. The vital information herein is measurement values of body temperature, pulse, systolic blood pressure, diastolic blood pressure, pulse pressure, and respiratory rate.

At any determination time point, when determining the vital information of the subject, the vital mean value μ and the vital standard deviation σ are calculated from the data of the same subject recorded in the information recording unit 4 using the above equations (3) and (4), starting from the day before the determination time point or the determination time point. That is, the scoring reference information 102 and the vital determination reference information 102a are calculated by a pattern without including the value of the vital sign which is the target of the determination measured at the determination time point or a pattern including the value of the vital sign which is the target of the determination measured at the determination time point.

In addition, the scoring reference setting means 101 and the vital determination reference configuring means 101a use the values expressed by the following equation (1) or (2) as the scoring reference information 102 and the vital determination reference information 102a:

μ−nσ Equation (1); and

μ+mσ Equation (2).

Here, n and m are numbers greater than zero.

In addition, in the scoring reference information 102, the value shown by said Equation (1) and Equation (2) and the predetermined score value, i.e., the information of the score of 0-3 points are combined. This combination is, for example, as shown in Table 3 below.

TABLE 3

| Score | 3 | 2 | 1 | 0 | 1 | 2 | 3 |
|---|---|---|---|---|---|---|---|
| Systolic Blood | −3σ< | −3σ~−2.5σ | −2.5σ~−2σ | Within ±2σ | +2σ~+2.5σ | +2.5σ~+3σ | +3σ> |
| Pressure (mmHg) | | | | | | | |
| Diastolic blood | −3σ< | −3σ~−2.5σ | −2.5σ~−2σ | Within ±2σ | +2σ~+2.5σ | +2.5σ~+3σ | +3σ> |
| pressure (mmHg) | | | | | | | |
| Pulse pressure | −3σ< | −3σ~−2.5σ | −2.5σ~−2σ | Within ±2σ | +2σ~+2.5σ | +2.5σ~+3σ | +3σ> |
| (mmHg) | | | | | | | |
| Pulse rate | −3σ< | −3σ~−2.5σ | −2.5σ~−2σ | Within ±2σ | +2σ~+2.5σ | +2.5σ~+3σ | +3σ> |
| (beats/minute) | | | | | | | |
| Body | −3σ< | −3σ~−2.5σ | −2.5σ~−2σ | Within ±2σ | +2σ~+2.5σ | +2.5σ~+3σ | +3σ> |
| temperature | | | | | | | |
| (° C.) | | | | | | | |
| Oxygen | 84 or less | 85~89 | 90~92 | 93~100 | — | — | — |
| Saturation (%) | | | | | | | |
| Respiratory | Mode −10< | Mode −6~−9 | Mode −5 | Within Mode ±4 | Mode +5 | Mode +6~9 | Mode +10> |
| rate (number of | | | | | | | |
| breaths/minute) | | | | | | | |
| Consciousness | Unconsciousness | Response | abnormal | normal | — | — | — |
| Level | | to pain | | | | | |

In addition, in Table 3 and Table 4 below, “−3σ” is a value of “μ−3σ” based on equation 1, “−2.5σ” is a value of “μ−2.5σ” based on equation 1, “−2σ” is a value of “μ−2σ” based on equation 1, “+3σ” is a value of “μ+3σ” based on equation 2, “+2.5σ” is a value of “μ+2.5σ” based on equation 2, and “+2σ” is a value of “μ+2σ” based on equation 2. In addition, μ and σ are the values computed from the measurement value of each vital sign measured on predetermined conditions (for example, vital information including at least 4 pieces). As shown in Table 3, when scoring the measurement values of body temperature, pulse, systolic blood pressure, diastolic blood pressure, and pulse pressure, based on the contents thereof, the “μ±2σ, μ±2.5σ and μ±3σ” are calculated based on the equations (1) and (2).

More specifically, if the measurement value of the input vital sign is, in the vital mean value and the vital standard deviation calculated at the determination time point, a value belonging to the range of “within μ±2σ”, it will be a score of 0 point, a value belonging to a range of “μ−2.5σ (or greater)˜(under) μ−2.5σ” or a range of “μ+2σ (or greater)˜(under) μ+2σ”, it will be a score of 1 point, a value belonging to a range of “μ−3σ(or greater)˜(under) ∥−2.5σ” or a range of “μ+2.5σ(exceed)˜(within) μ+3σ”, it will be a score of 2 points, a value belonging to a range of “(under) μ−3σ” or “μ+3σ(exceeds)”, it will be a score of 3 points.

In addition, unlike Table 3, in the content shown in Table 4 below, the values indicated in the above equations (1) and (2) and a predetermined score value, that is, the information of a score of 0 to 2 points can be combined as the scoring reference information 102.

TABLE 4

| Score | 2 points | 1 point | 0 point |
|---|---|---|---|
| Body | ±3σ or greater | ±2σ~±3σ | Within ±2σ |
| temperature | | | |
| (° C.) | | | |
| Systolic | ±3σ or greater | ±2σ~±3σ | Within ±2σ |
| blood | | | |
| pressure | | | |
| (mmHg) | | | |
| Diastolic | ±3σ or greater | ±2σ~±3σ | Within ±2σ |
| blood | | | |
| pressure | | | |
| (mmHg) | | | |
| Pulse | ±3σ or greater | ±2σ~±3σ | Within ±2σ |
| pressure | | | |
| (mmHg) | | | |
| Pulse | ±3σ or greater | ±2σ~±3σ | Within ±2σ |
| (beats/minute | | | |
| Respiratory | ±3σ or greater | ±2σ~±3σ | Within ±2σ |
| rate (number of | | | |
| breaths/minute) | | | |
| Oxygen | — | 93% or less | 94% or greater |
| saturation | | | |
| (%) | | | |
| consciousness | Unconscious, | Abnormal | Normal |
| level | abnormal | | |
| | state, dazed, | | |
| | hazy | | |
| Symptom | 2^{nd} item | 1^{st} item | 0^{th} item |

\textcircled{1}general boredom \textcircled{2}anorexia
\textcircled{3}shortness of breath \textcircled{4}Zyanose (purple lips)
\textcircled{5}skin tension (turgor) \textcircled{6}cough

In addition, the contents shown in Tables 3 and 4 are examples of the scoring reference information 102, and the contents of the combination of the values indicated in the above Equations (1) and (2) and the predetermined score value are not limited to the contents of Tables 3 and 4, and other configuring can be made. As for scoring of the measurement value of the input vital sign, the reference for each determination time point is configured by the vital mean value and the vital standard deviation calculated at the determination time point. In addition, the measurement values of body temperature, pulse rate, systolic blood pressure, diastolic blood pressure, pulse pressure, and respiratory rate are vital signs according to a normal distribution, and the scoring reference information 102 calculated based on Equations (1) or (2) is a reference in which the subject's individual variation is reflected. Therefore, it becomes an index which can determine the variation of the condition of a subject correctly.

Further, for example, the determination processing means 6 determines the value of “μ±2σ or more” which is equal to or more than (the value of the vital sign) with respect to the value of the vital sign (measurement value of each vital sign) as “abnormality (the value of the vital sign)”. That is, in this case, as the vital determination reference information 102a, a value of “μ±2σ or more” becomes the reference for determining the presence or absence of abnormality.

Moreover, the determination processing means 6 designates “caution” when 1 point is calculated with respect to the score value information 103, and designates “warning” when 2 or more points are calculated. When the score value information 103 is 0, the determination result of “caution” or “warning” does not appear, and it can be seen as a “normal” state. In other words, when a determination is made that the measurement value of each vital sign is a value of 1 or more points, the measurement can be made as an abnormality divided into two stages of “caution” and “warning”. This content is score determination reference information 18.

Further, the score value information 103 calculated from the values of the vital signs, the score determination result information 12 such as attention to this value, and the vital determination result information 12a are recorded in the information recording unit 4 in association with the subject.

In addition, when the determination processing means 6 has made the determination of “warning” for the score value information 103 or the determination of “warning” for the value of the vital sign, the health condition management device 1 may generate an alarm sound or transmit an e-mail indicating that a “warning” has been made through the information transmission/reception unit 3c. Thereby, the caregiver etc. can be notified that an abnormality has occurred in the condition of the subject. In addition, here, the determination of the score value information 103 may be mainly configured to generate an alarm sound or transmit an e-mail to an external terminal or the like when the report on the determination of the “warning” for the score value information 103 is performed and the “warning determination” is performed.

Here, although n in the above Equation (1) or (2) has been described as being greater than 0, the numerical values of n and m are not limited to “2, 2.5 and 3” as described above, and the numerical value can be changed suitably and can be used as the scoring reference information 102 or the vital determination reference information 102a.

In addition, the measurement values of body temperature, pulse, systolic blood pressure, diastolic blood pressure, pulse pressure, and respiratory rate do not necessarily need to have the same as n and m in Equation (1) or Equation (2). Depending on the type of vital sign, the numerical values to be set to n and m may be different.

In addition, in the scoring reference information 102 shown in Table 3, as a range which distinguishes the score value information of 1 point and the score value information of 2 points, for example, the range of “within μ±2σ” and “μ+2σ (or greater)˜(less than) μ+2.5σ” is configured. That is, before and after the numerical value of μ+2σ, if within 0 μ+2σ, it becomes 0 points, and if beyond 0 μ+2σ, it becomes 1 point. However, the configuring of the range is not necessarily limited to this content. For example, it can also be set as the content which becomes 0 points for less than μ+2σ, and one point for μ+2σ or greater. In addition, the same applies to other numerical values.

In addition, in the scoring reference information 102 shown in Table 3, although the score value information 103 is configured in the range of 0 to 3 points (configured in the range of 0 to 2 points in Table 4), it is not necessarily limited to this range. For example, it is also possible to change the score value information into a setting that scores in a range of 0, 1 and 2 points. Furthermore, it is also possible to employ a value larger than three points. It goes without saying that the scoring reference information 102 can be set appropriately to be suitable when the score value information 103 is changed. This also applies to scoring of oxygen saturation and consciousness level described later.

In addition, the numerical value which the determination processing means 6 determines to be abnormal about the score value information 103 is not limited to one or more points. For example, a determination using two points or more may be adopted. In addition, it is not necessary to determine the above determination in two stages of “caution” and “warning.” For example, there may be a configuration for dividing the determination into three or more stages, or an aspect for designation simply as one stage of “abnormality”. However, it is possible to distinguish the degree of abnormality of the score value information 103 by determining the above determination in two stages of “caution” and “warning”, so that the countermeasures according to the degree of “caution” or “warning” are easily set. It is preferable to divide the above determination into two stages. This also applies to scoring of oxygen saturation and consciousness level described later.

In addition, although the determination processing means 6 is set to perform determination of whether it is an abnormal value with respect to the score value information 103 based on the measurement value of each vital sign, it does not necessarily need to be set in this way. For example, it may be set as an aspect which determines whether it is an abnormal value with respect to the “total points” of the score value information 103 based on multiple types of vital signs.

For example, it may be set as an aspect in which the score determination reference information 18 for determining whether it is abnormal with respect to the “total score” of the score value information 103 of all types of vital signs is set, and determination on whether the value is abnormal for the “total points” of each score value information 103 is performed. In addition, it is also possible to provide an aspect in which specific types of vital signs (e.g., body temperature and pulse rate) are combined to determine whether the value is abnormal for the “total points” of the score value information 103 based on the combined vital signs.

In addition, for example, “caution” or “warning” is configured for the “total point” of the score value information 103 based on a plurality of types of vital signs according to the score, and this “caution” or “warning” may be set to be displayed on the display screen 3b or to cause alert to ring.

In addition, in the scoring reference information 102 shown in Table 3 and Table 4, systolic blood pressure, diastolic blood pressure, pulse pressure, pulse, body temperature, respiratory rate, oxygen saturation level, and consciousness level can be cited as targets to be scored (markers), this is merely one example. In addition, the threshold value which distinguishes the score in scoring reference information 102 is also only an example.

That is, the threshold value for distinguishing the type or score of the marker can be configured differently according to the type of the disease or the nature of the subject. For example, in the case of a subject having heart failure and a subject having a urinary tract infection, the threshold value for distinguishing the type or score of the marker may be configured differently. Further, as the marker, there may be a case where only systolic blood pressure is employed in the blood pressure or both systolic blood pressure and diastolic blood pressure are employed. In addition, for example, in the case of a subject who is a normal person and a subject who is an elderly person having a disease, the threshold value for distinguishing the type or score of a marker may also be configured differently.

In addition, the scoring reference information 102 also includes an aspect of scoring, including markers such as a history of the subject, a disease-related family history of the subject's family and relatives, and lifestyle.

In this case, for example, when performing scoring to determine the degree of heart failure for a subject with a history of heart disease or a subject having a history of heart disease in their family, a score is given to a marker of history or family history and score is added to the total points of the value information 103. In addition, for example, for a subject who has a lifestyle of smoking, a score is given to the lifestyle marker and is added to the total points of the score value information 103.

Here, the difference between the case where the distribution of vital information based on the information of another individual is established using the vital information of a plurality of subjects and the case where the distribution of vital information of the same individual is established using the vital information of the same subject will be explained.

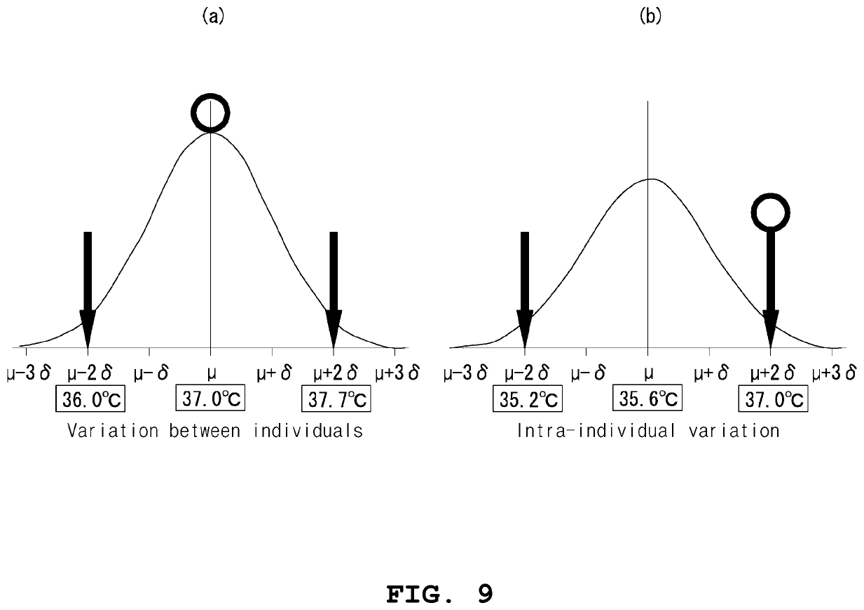

FIGS. 9A and 9B are graphs of a normal distribution curve established based on body temperature information. In FIGS. 9A and 9B, the horizontal axis represents a probability variable of body temperature, and the vertical axis represents a probability density. FIG. 9A is made up of many subjects, and FIG. 9B is made up of only the same subjects. In FIG. 9A, persons with various normal temperatures and body temperature fluctuations are included, and the mean value μ is 37.0° C., which is the mean value of a plurality of subjects, the value of μ+2σ is 37.7 ° C., and the value of μ−2σ is 36.0° C.

However, in FIG. 9B, the vital information of the same individual is recorded, and since the normal temperature and body temperature peculiar to the person vary, the mean value μ is 35.6° C., the value of μ+2σ is 37.0° C., and the value of μ−2σ is 35.2° C.

That is, if the reference value stabilized to a certain score value at the time of scoring using each distribution is configured to μ+2σ, in the case of FIG. 9A, the body temperature of 37.0° C. corresponds to the position of p (black circle in FIG. 9A). On the other hand, in FIG. 9B, the body temperature at 37.0° C. becomes the position of the upper limit of μ+2σ (black circle in FIG. 9B).

That is, in the distribution shown in FIG. 9A and the distribution shown in FIG. 9B, the values of the same μ+2σ on the distribution become completely different value. Therefore, the vital determination reference information 102a, the scoring reference information 102, and the score value information 103 are also changed, and the determination result is also changed.

In other words, in performing the determination of the subject of FIG. 9B, the vital determination reference information 102, the scoring reference information 102a, or the score value information 103, based on the vital information of the plurality of subjects cannot be used to determine an “abnormal value”. Using the vital information of a large number of persons as a reference is not only a determination in the “inter-individual variation” which has been conventionally performed, but it shows that the “intra-individual variation” is effective in order to see the variation of the vital information peculiar to the subject.

In addition, the subject who exhibits the mean value or variation of the body temperature shown in FIG. 9B does not correspond to a special case. In addition, a phenomenon is caused only by body temperature, and the variation unique to the subject may also occur in other vital signs such as systolic blood pressure, diastolic blood pressure, pulse rate, and respiratory rate, and these are subject to normal distribution. As an example of the above-mentioned body temperature, there are many elderly people whose body temperature changes in the temperature range shown in FIG. 9B, and “intra-individual variation” is effective when performing the determination of the health condition of the elderly by using the vital signs.

### [7-2. About Measurement Value of Oxygen Saturation]

As a configuration method of the scoring reference information 102 about the measurement value of the oxygen saturation measured from the subject, the information of a certain numerical range is configured as a reference. In the content shown in Table 3, when scoring with the score value of 0-3 points with respect to the measurement values of oxygen saturation degree, “93-100 (%)” is configured as a score of 0 point, “90-92 (%)” is configured as a score of 1 point, “85-89 (%)” is configured as a score of 2 points, and “84 (%) or less” is configured as a score of 3 points.

The score value information 103 of 0-3 points is calculated based on the scoring reference information 102 shown in Table 3 about the measurement value of oxygen saturation input. In addition, the determination as to whether it is an abnormal value by the determination processing means 6 for the score value information 103 is as described above.

In addition, score value information 103 calculated from the measurement value of oxygen saturation and score determination result information 12 such as attention to this value are recorded in the information recording unit 4 in association with the subject.

Here, the content of the scoring reference information 102 for the oxygen saturation degree shown in Table 3 and Table 4 is not limited thereto. The numerical range which divides the score value information of 0-3 points can be set as the scoring reference information 102 by suitably changing a configuration.

### [7-3. About Measurement Value of Respiratory Rate]

As a method of setting the scoring reference information 102 for the measurement value of the respiratory rate measured from the subject includes an aspect using a value of “μ±nσ”, as shown in Table 4.

As another method of setting the scoring reference information 102 for the measurement value of the respiratory rate measured from the subject, a method of using the vital information 8 and the re-measured vital information 13 recorded in the information recording unit 4 for the calculation of the mode can be mentioned. In this method, the mode calculation means calculates the mode with respect to the measurement value of the respiratory rate under predetermined conditions (e.g., 30 minutes). In addition, the measurement value of respiratory rate can adopt the value of the respiratory rate measured on the conditions set. In addition, the content of all vital information here may extract a part of the information recorded in the information recording unit 4 as mentioned above.

At any determination time point, when determining the respiratory rate of the subject, starting from the determination time point, the mode is calculated from the data of the same subject recorded in the information recording unit 4. That is, the scoring reference information 102 is calculated at the determination time point. The scoring reference configuring means 101 configures the scoring reference information 102 from the mode so as to be the contents shown in Table 3.

The mode is calculated with respect to the measurement value of the input respiratory rate, and based on this mode, it becomes the scoring reference information 102 shown in Table 3, and the score value information 103 of 0-3 points is computed. In addition, the determination as to whether or not it is an abnormal value by the determination processing means 6 for the score value information 103 is as described above.

### [7-4. About Consciousness Level]

A caregiver or the like may check the consciousness level of the subject and apply the acquired result to predetermined observation information set as the scoring reference information 102. Confirmation of consciousness level may use existing AVPU assessments.

In the AVPU evaluation, Normal (Awake alert, A: alert), Abnormal (Respond by words but no orientation, V: verbal), Respond to pain (Respond only to pain, P: Pain), and Unconsciousness (not responding to words or pain, U: Unresponsive) are set as predetermined observation states. A caregiver or the like observes the subject to determine which item of the AVPU evaluation the consciousness level corresponds to, and inputs the result through the input unit 3a or the like.

Scoring reference information 102 for the consciousness level is configured, for example, by the content shown in Table 3. In Table 3, a normal score is 0, abnormality is a score of 1, painlessness is a score of 2 points, and unconsciousness is set to a score of 3 points. The scoring processing means 100 calculates the score value information 103 based on the information input by the caregiver or the like. In addition, the determination as to whether or not it is an abnormal value by the determination processing means 6 for the score value information 103 is as described above.

Here, the content of the scoring reference information 102 regarding the evaluation result of the consciousness level of the subject shown in Table 3 (or Table 4) is not limited to this. Consciousness level evaluation techniques other than AVPU evaluation may be employed. In addition, the observation state which divides the score value information of 0-3 points may be suitably changed, and can be configured as the scoring reference information 102.

In the above description, scoring is performed using the measurement values of the body temperature, pulse, systolic blood pressure, diastolic blood pressure, pulse pressure, oxygen saturation, and respiratory rate, and the evaluation result of consciousness level among vital signs of the subject, and whether the calculated score value information 103 is abnormal is determined. Here, the vital sign of the subject need not necessarily be limited to these contents. For example, as a subject to be scored, it is also conceivable to employ urine volume, weight, pain (with or without pain), and other pathological abnormalities obtained from the subject as vital sign information.

In addition, in the above content, both the configuration for performing “abnormality determination on the value of vital signs” and the configuration for performing “scoring from the value of vital signs and abnormality determination on the scored score value” are included. However, in the present disclosure, the two configurations are not necessarily combined. That is, as an aspect of the present disclosure, only the invention of a configuration that performs “abnormality determination on the value of vital signs” and the invention of a configuration that performs “scoring from the value of vital signs and abnormality determination on the scored score value” may exist separately.

### [8. Creation of Display Information]

In the health condition determination device 1 to which the present disclosure is applied, the content of the subject's vital information can be displayed as a normal distribution curve. It is also possible to display the vital information of the subject as a thermal table.

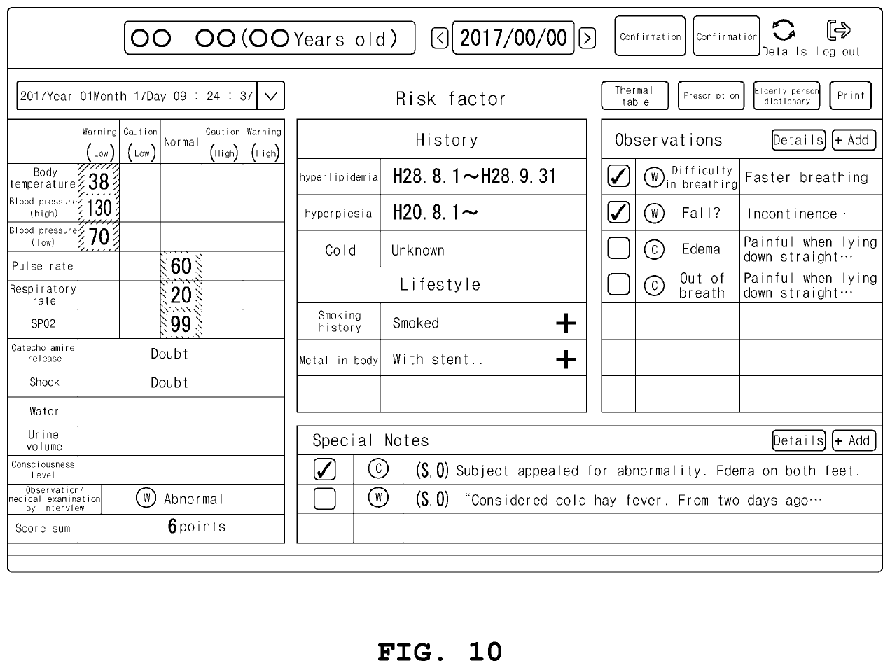

FIG. 10 shows an example of the thermal table. FIG. 10 shows vital information at the time of determination concerning a subject, information on whether the value of the score value information based on the content of the vital information is an abnormal value (warning, caution, normal information), information on the presence or absence of abnormality due to the observation or questionnaire result of the subject, and information of total points of sore value information.

In addition, in the thermal table shown in FIG. 10, the information on the past history, which is a risk factor of the health condition of the subject, and the information on lifestyle are displayed. In addition, the thermal table displays detailed observation information of the subject and information on special items. The information displayed on the thermal table can be created based on the information input via the input unit 3a or the like.

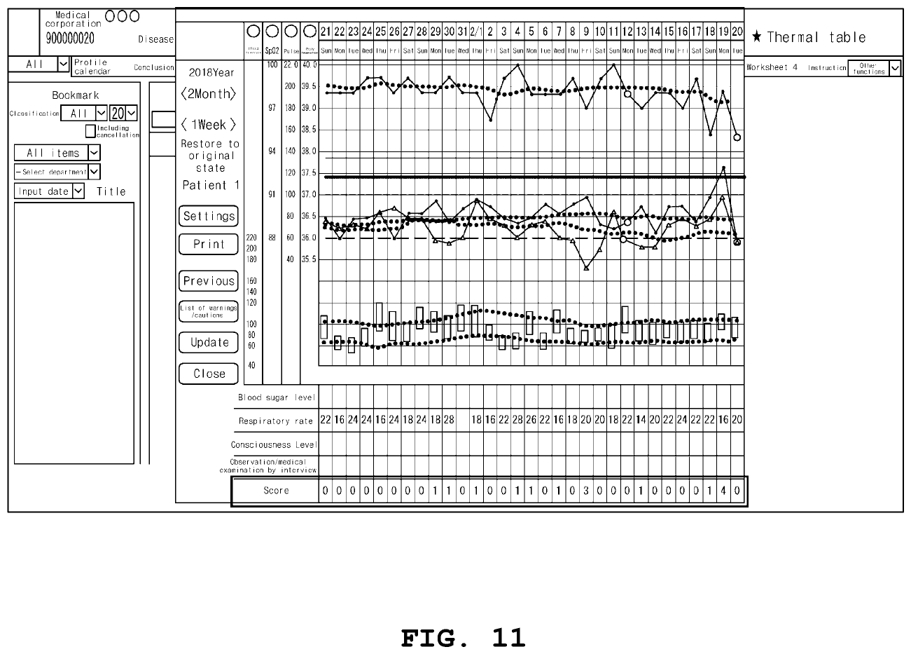

In addition, FIG. 11 shows an image showing the value of the score value information based on the content of the vital information in the thermal table, which is one of the display information of the electronic chart, in the electronic chart used in the terminal installed in the hospital or the like. For example, the aspect of totaling a sum of the score values of several vital information and displaying the sum value of the score values for every day can be considered. In this case, the information based on the result of the scoring together with the information of the electronic chart in which the patient information is recorded can be used for the risk assessment of the subject.

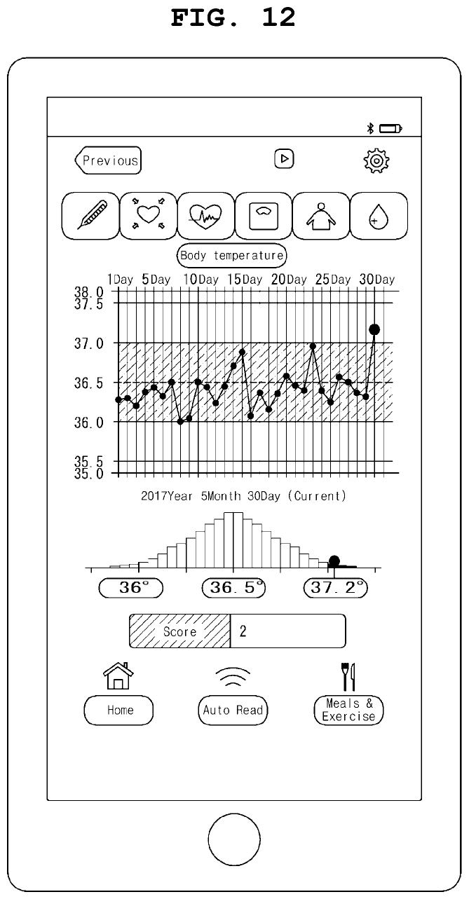

Further, FIG. 12 shows an image showing the value of the score value information based on the content of the vital information on the screen when the application software having the function of the software of the present disclosure is used in a smartphone terminal or the like. For example, there may be an aspect that indicates the recording (temperature) of vital information of the user of the smartphone terminal and the value of the score value information. In this case, the information based on the result of the scoring can be utilized for the health management in the smartphone or the evaluation of the condition of health in the home health care.

[9. Determination of Measurement Accuracy with or without Normal Distribution and Determination of Abnormal Values]

In the health condition determination device 1 to which the present disclosure is applied, the Q-Q plot can be used as a technique for checking whether the measured vital information is suitable for a normal distribution. For example, the vital standard deviation of the subject is plotted by taking the value of the vital standard deviation on the horizontal axis and the value of the percent point of the standard normal distribution corresponding to the cumulative probability of the standard deviation on the vertical axis. If each plot is located on a straight line, it is possible to visually confirm that the acquired vital information is normally distributed.

Next, a series of flows of information processing in the software to which the present disclosure is applied will be described with reference to drawings.

### [Abnormality Determination of Vital Signs]

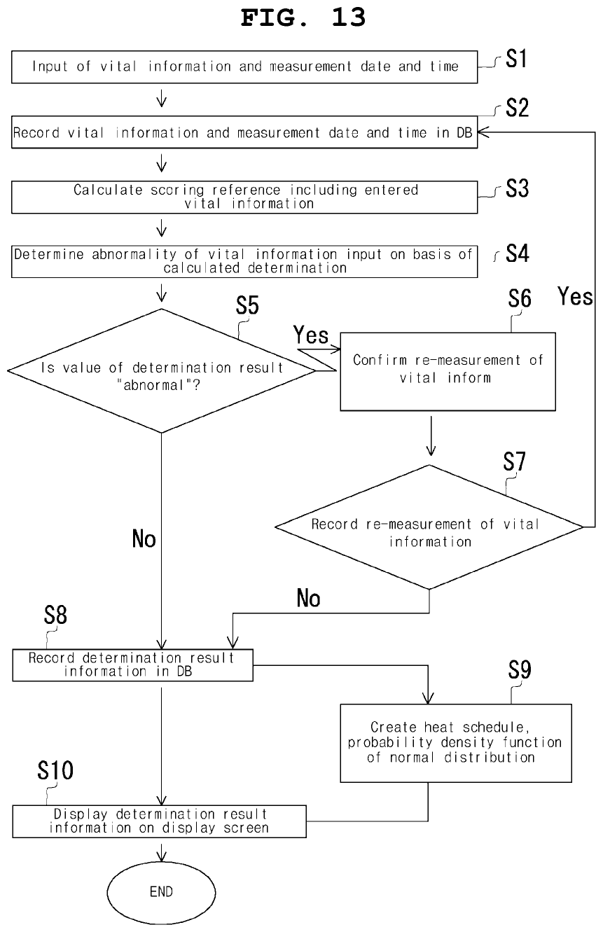

FIG. 13 shows the flow of information processing from input of vital information to abnormality determination and display of resultant information. First, the value of the subject's vital signs is measured by each measuring device, and the measurement values and information on measurement date and time are input. (S1). The input information is recorded in the information recording unit 4 (DB) as vital information of the subject (S2).

The calculation unit 2 functions as the reference calculation means 5, including vital information that is the subject of the determination recorded in the information recording unit 4, to calculate the determination reference (S3). Here, a vital mean value and a vital standard deviation are calculated, and based on these values, the determination reference (e.g., the upper or lower limit) under the configured conditions is created. That is, the determination reference is calculated at each determination time.

Subsequently, the determination on whether it is abnormal value based on the determination reference is performed for the input vital information of the subject to be determined (S4). As not being determined to be an “abnormal value” as the result of the determination, the determination result information is recorded in the information recording unit 4 (DB) (S8), and the information of the determination result is displayed on the display screen 3b (S10). Further, based on the subject's vital information, a thermal table that graphs changes over time in the value of the vital sign and the established density function of the normal distribution (graph of the normal distribution curve) are created as display information (S9), and this information can also be confirmed on the display screen 3b.

In addition, determination on whether it is an abnormal value is performed with respect to the input vital information of the subject to be determined, based on the determination reference (S4). As being determined to be an “abnormal value” as the result of the determination for example, the display screen 3b displays the indication “Do you wish to perform re-measurement?” or recommends attention to the posture at the time of acquisition of the vital signs, and then may confirm to the subject whether there is re-measurement of vital information (S6).

Here, when the subject selects “no re-measurement of vital information”, the determination result information of abnormality determination is recorded in the information recording unit 4 (DB) (S8), and the information of the determination result is displayed on the display screen 3b (S10). A thermal table or an established density function of the normal distribution (graph of the normal distribution curve) is created as display information (S9), and this information can also be confirmed on the display screen 3b.

In addition, if the subject selects “Perform re-measurement of vital information”, it prompts the input of the value of the remeasured vital sign and the measurement date and time, and the information in which the input remeasurement vital information is input is recorded in the information recording unit 4 (DB) as re-measurement vital information of the subject (S2). After that, calculation (S3) of determination reference and abnormality determination (S4) again are performed. In the determination, if the determination is not an abnormal value, the determination result information is recorded in the information recording unit 4 (DB) (S8). In addition, when it is determined that it is an abnormal value, it may proceed to the step of confirmation of presence or absence of re-measurement vital information (S6), or it may proceed to recording of determination result information (S8) as it is a 2nd determination result.

When the subject confirms the information of the determination result on the display screen 3b, a series of information process is completed. In the above-described flow, the software to which the present disclosure is applied performs the determination of the health condition through the vital information.

### [Abnormality Determination Based on Vital Scoring]

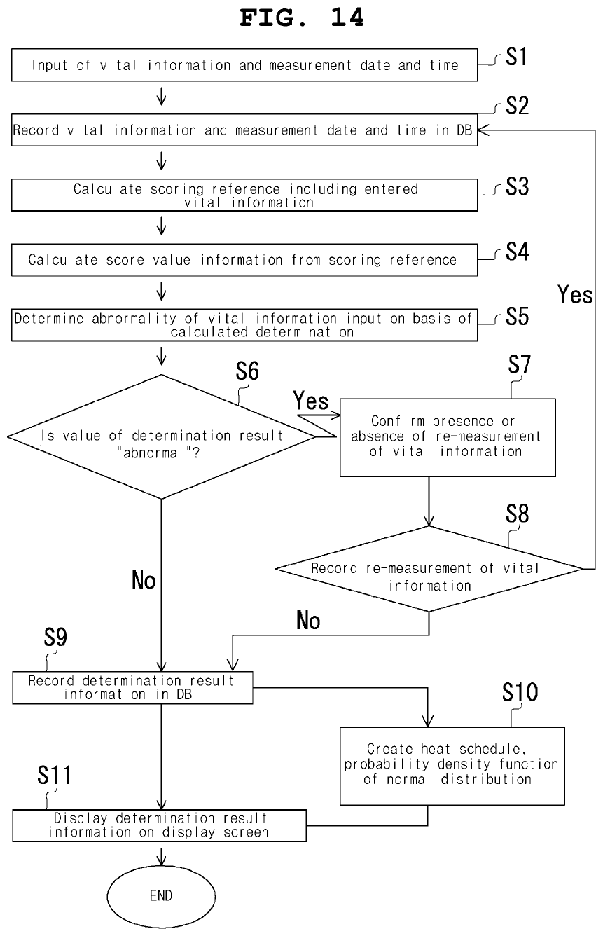

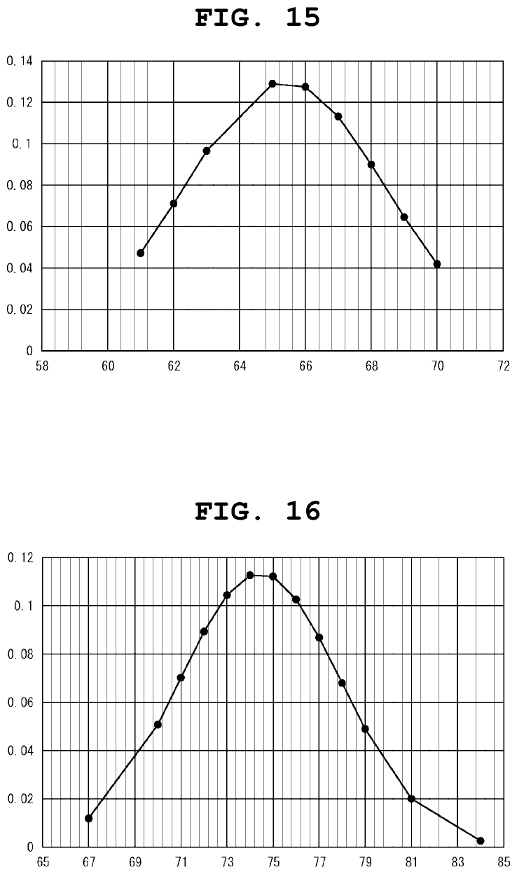

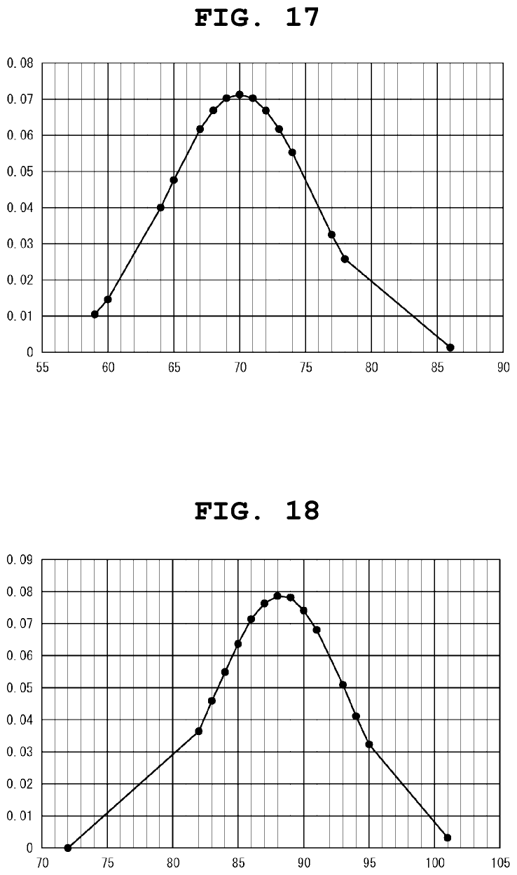

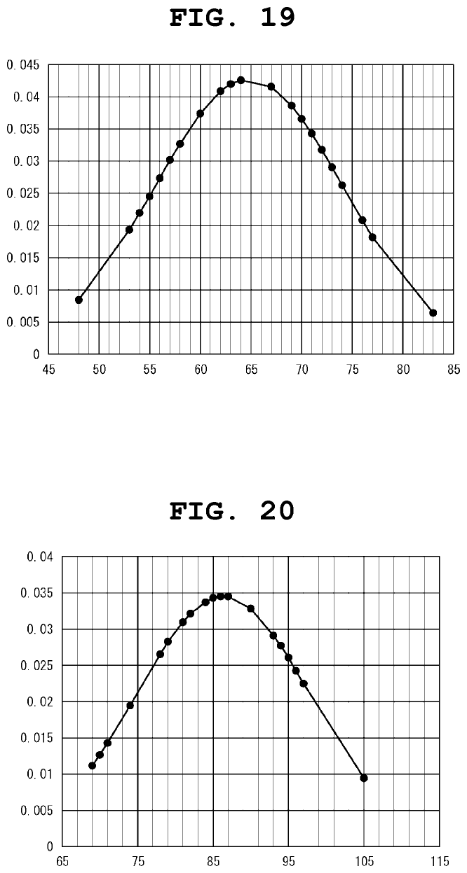

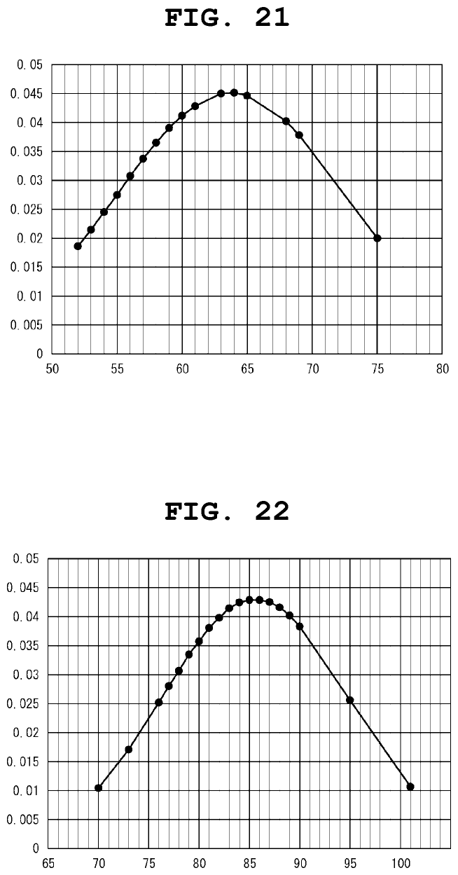

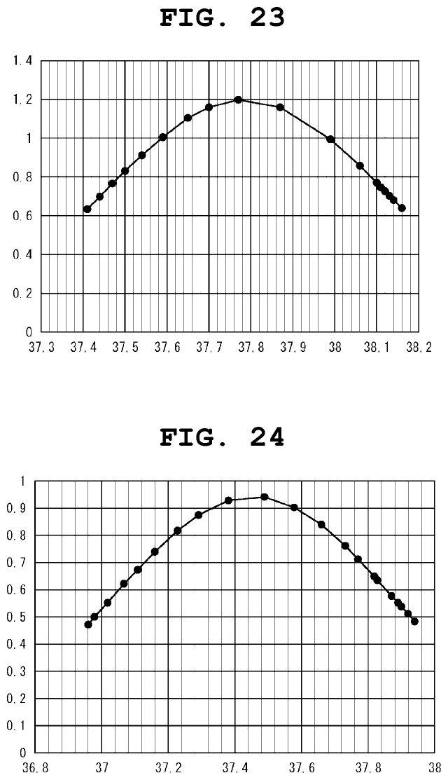

FIG. 14 shows the flow of information processing from input of vital information to abnormality determination in score value information and display of resultant information.

First, the value of the subject's vital signs (temperature, pulse, systolic blood pressure, diastolic blood pressure, pulse pressure, oxygen saturation, and respiratory rate) is measured by each measuring device, and the measurement values and information on date and time of measurement are input. (S1). At this time, the corresponding information of the observation information of the vital reference information 102 is selected or input from the evaluation result of the subject's consciousness level. The input information is recorded in the information recording unit 4 (DB) as vital information of the subject (S2).

The calculation unit 2 functions as the reference calculation means 5, including vital information that is the subject of the determination recorded in the information recording unit 4, to calculate the scoring reference information 102 (and to determine the vital determination reference information 102a) (S3). Here, a vital mean value and a vital standard deviation are calculated, and based on these values, the scoring reference information (a predetermined numerical range, etc.) under the configured conditions are created. Here, the references relating to body temperature, pulse, systolic blood pressure, diastolic blood pressure, pulse pressure, and respiratory rate are calculated at each time of scoring (and upon determination of the value of the vital signs).

Subsequently, the score value information 103 is calculated for each vital information by the scoring processing means 100 based on the scoring reference information 102 with respect to the input vital information of the subject to be determined.

When the score value information 103 is calculated, the determination processing means 6 determines whether the score value information is an abnormal value based on the determination reference (S5). As not being determined to be an “abnormal value (caution or warning)” as the result of the determination, the determination result information is recorded in the information recording unit 4 (DB) (S9), and the information of the determination result is displayed on the display screen 3b (S11). Further, based on the subject's vital information, a thermal table that graphs changes over time in the value of the vital sign and the established density function of the normal distribution (graph of the normal distribution curve) are created as display information (S10), and this information can also be confirmed on the display screen 3b.

In addition, based on the reference of determination on the input vital information of the subject to be determined, as being determined to be an “abnormal value (caution or warning)” as the result of the determination, for example, the display screen 3b may display the indication “Do you wish to perform re-measurement?” or recommend attention to the posture at the time of acquisition of the vital signs, and then may confirm to the subject whether there is re-measurement of vital information (S7).

Here, when the subject or the caregiver selects “no re-measurement of vital information”, the determination result information with the abnormality determination is recorded in the information recording unit 4 (DB) (S9), and the information of the determination result is displayed on the display screen 3b (S11). Further, a thermal table or an established density function of a normal distribution (graph of a normal distribution curve) is prepared as display information (S10), and this information can also be confirmed on the display screen 3b.

In addition, if the subject or the caregiver selects “Perform re-measurement of vital information”, it prompts the input of the value of the remeasured vital sign and the measurement date and time, and the information in which the input remeasurement vital information is input is recorded in the information recording unit 4 (DB) as re-measurement vital information of the subject (S2). After that, calculation (S3) of scoring reference information and abnormality determination (S4) of the score value information again are performed. In the determination, if the determination is not an abnormal value, the determination result information is recorded in the information recording section 4 (DB) (S9). In addition, when it is determined that it is an abnormal value, it may proceed to the step of confirmation of presence or absence of re-measurement vital information (S6), or it may proceed to recording of determination result information (S9) as it is a 2nd determination result.

In addition, although details are not shown in FIG. 14, the determination processing means 6 determines whether the value of the input vital sign is an abnormal value, based on the vital determination reference information 102a. For the result of the determination, the determination result information is recorded in the information recording unit 4 (DB), and the information of the determination result is displayed on the display screen 3b as not being determined to be an “abnormal value (warning)”.

In addition, with respect to the input vital information of the subject of determination, when the value of the vital sign is determined to be an “abnormal value (warning)” as a result of the determination, based on the vital determination reference, for example, the display screen 3b displays, “Will you perform re-measurement?” or recommends attention to the posture at the time of the acquisition of the vital sign, and confirms to the subject whether there is re-measurement of vital information.

Here, when the subject or the caregiver selects “no re-measurement of vital information”, the determination result information of abnormal determination is recorded in the information recording unit 4 (DB), and the information of the determination result is displayed on the display screen 3b.

In addition, the information recording unit 4 records the value of the vital sign which has been determined as an abnormal determination to be included in the vital information 8. As a result, the vital information 8 accumulates both the vital information in which the score value information is determined to be a normal value and the vital information in which the score value information is determined to be an abnormal value. That is, not only the abnormality of the above-described score value information, but also the data of the abnormality of the value of the vital sign can be accumulated.

When the subject confirms the information of the determination result on the display screen 3b, a series of information process is completed. In the above-described flow, the software to which the present disclosure is applied performs the determination of the health conditions from the vital information.

Next, an example of performing abnormality determination (abnormality determination on vital signs) on the value of a vital sign will be described with reference to the drawings.

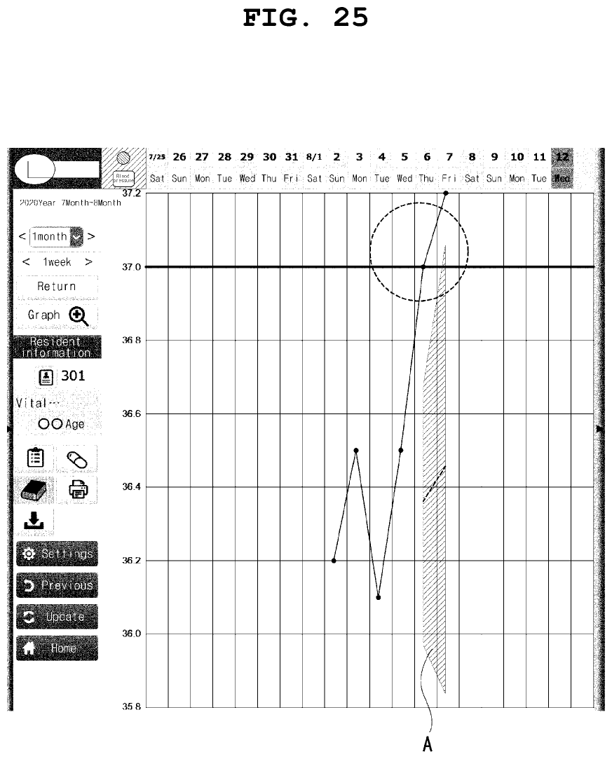

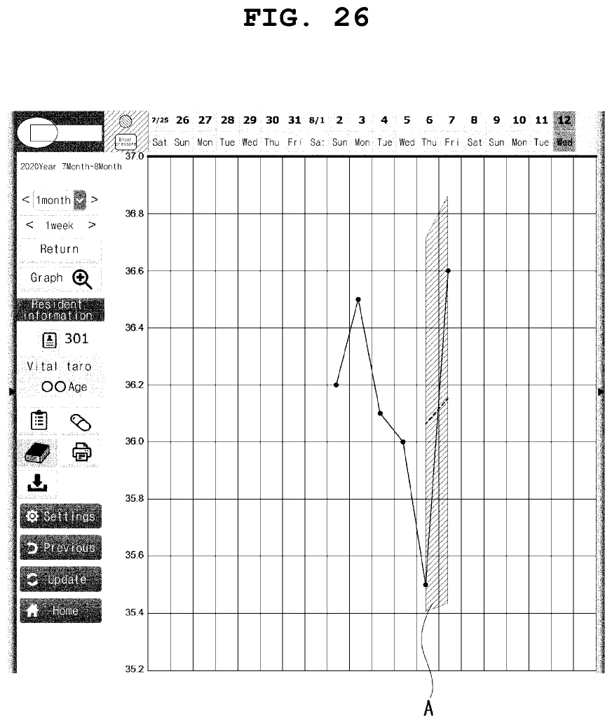

FIGS. 25 and 26 show cases in which abnormality determination of vital signs with respect to body temperature is performed using vital reference information based on vital information for 4 days or 5 days. Here, the value of body temperature measured once a day from August 2 to August 7 is shown in a broken line graph. In addition, the area indicated by symbol A is the range which shows the vital reference information at the time of determination on August 6 (5th day), and the vital reference information at the time of determination on August 7 (6th day). In the example shown in FIG. 25, the range of the vital reference information A at the time of determination on August 6 (5th day) is set as follows. Here, the vital mean value (μ) and vital standard deviation (o) are calculated based on the body temperature for 4 days from August 2 to August 5 without including the body temperature (37.0° C.) at the time of determination on August 6, and becomes the vital reference information A with “μ+2σ” as the upper limit and “μ−2σ” as the lower limit.

In addition, in the case shown in FIG. 25, the range of the vital reference information A at the time of determination on August 7 (6th day) is set as follows. Here, the vital mean value (μ) and vital standard deviation (σ) is calculated based on the body temperature for 5 days from August 2 to August 6 without including the body temperature (37.2° C.) at the time of determination on August 7, and becomes the vital reference information A with “μ+2σ” as the upper limit and “μ−2σ” as the lower limit.

Then, at the time of determination on August 6 (5th day), the result of the vital reference information A exceeding the range is obtained with respect to the body temperature (37.0° C.) on that day. Therefore, the determination result as being “abnormal” is made in the abnormality determination on the vital sign of the body temperature on August 6.

In addition, at the time of determination on August 7 (6th day), the result of the vital reference information A exceeding the range is obtained with respect to the body temperature (37.2° C.) on that day. Therefore, the determination result as being “abnormal” is made in the abnormality determination on the vital sign of the body temperature on August 7.

In the case shown in FIG. 26, the range of vital reference information A of body temperature is set similarly to the case shown in FIG. 25.

In the example shown in FIG. 26, at the time of determination on August 6 (5th day), a result of the vital reference information A being located within the range is obtained with respect to the body temperature (35.5° C.) on that day. Therefore, the determination result as being “normal” (no abnormality) is made in the abnormality determination on a vital sign of the body temperature on August 6.

In addition, at the time of determination on August 7 (6th day), a result of the vital reference information A being located within the range is obtained with respect to the body temperature (36.6° C.) on that day. Therefore, the determination result as being “normal” (no abnormality) is made in the abnormality determination on a vital sign of the body temperature on August 7.

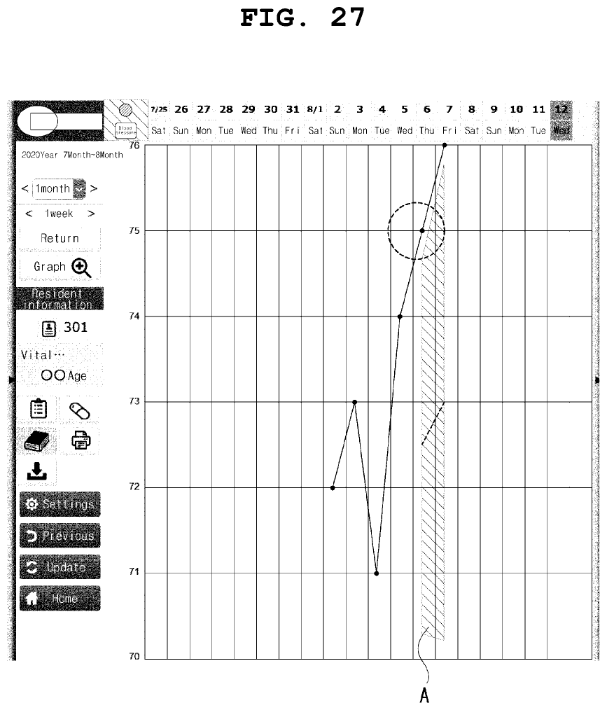

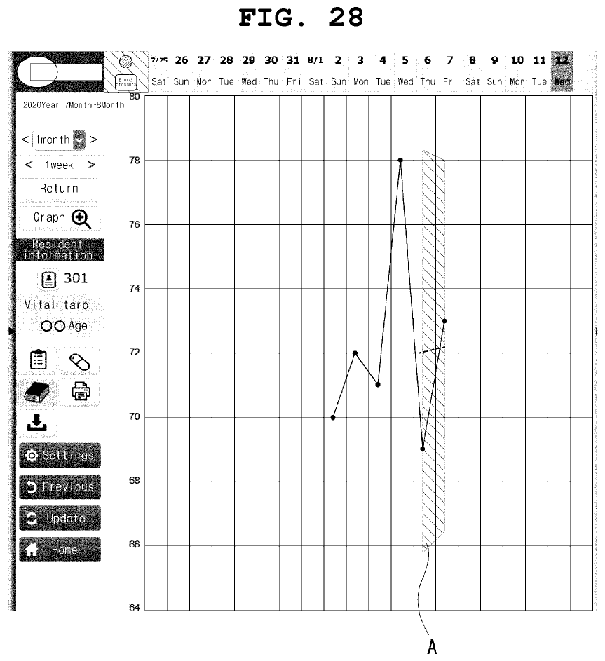

Moreover, an example of performing abnormality determination on a vital sign of pulse will be described with reference to FIGS. 27 and 28. In the case shown in FIGS. 27 and 28, the range of vital reference information A of pulse is set similarly to the cases shown in FIGS. 25 and 26.

In addition, in the case shown in FIG. 27, at the time of determination on August 6 (5th day), a result of vital reference information A exceeding the range is obtained with respect to the pulse (75 beats/min) on that day. Therefore, the determination result as being “abnormality” is made in the abnormality determination on the vital sign of the pulse on August 6.

In addition, at the time of determination on August 7 (6th day), a result of vital reference information A exceeding the range is obtained with respect to the pulse (76 beats/min) on that day. Therefore, the determination result as being “abnormality” is made in the abnormality determination on the vital sign of the pulse on August 6.

On the other hand, in the case shown in FIG. 28, at the time of determination on August 6 (5th day), result of the vital reference information A being located within the range is obtained with respect to the pulse (69 beats/minute) on that day. Therefore, the determination result as being “normal (no abnormality)” is made in the abnormality determination on the vital sign of the pulse on August 6.

In addition, at the time of determination on August 7 (6th day), the result of the vital reference information A being located within the range is obtained with respect to the pulse (73 beats/minute) on that day. Therefore, the determination result as being “normal (no abnormality)” is made in the abnormality determination on the vital sign of the pulse on August 7.

As described above, in the abnormality determination of vital sign using the present disclosure, vital information for a very short period is acquired, and vital reference information reflecting intra-individual variation of the subject is generated, so that determination on whether the value of the vital sign is abnormal can be made.

In addition, in the case shown in FIGS. 25 to 28, an aspect in which the vital reference information is set without including the vital information at the time of determination is provided, but in the present disclosure, an aspect in which the vital information at the time of determination is included and the vital reference information is set may be provided.

In addition, the present disclosure may employ both an aspect in which in relation to abnormality determination of the vital signs, vital information having the value of the vital sign determined as “abnormality” is included and the vital reference information is set, and an aspect in which in relation to abnormality determination of the vital signs, vital information having the value of the vital sign determined as “abnormality” is not included and the vital reference information is set.

In addition, although the cases shown in FIGS. 25 to 28 provide only two at the time of determination on August 6 (5th day) and at the time of determination on August 7 (6th day), in the present disclosure, for example, the records of the vital information may be accumulated after August 8 (after 7th day) and the generation of vital reference information and the abnormality determination of vital sign may be continuously performed.

In addition, when vital information is accumulated, all or part of the vital information recorded in the information recording unit may be extracted to generate vital reference information.

For example, based on a predetermined test method, an aspect in which measurement values of vital signs that can be determined as values for which normality is not guaranteed are excluded as values that can be considered abnormal as values of vital signs, and only quality data with guaranteed normality is extracted and used for abnormality determination of the vital signs may be provided.

Here, as a technique for determining normality, for example, the Shapiro-Wilk test can be employed. Shapiro-Wilk's test calculates the P value for a set of measured values of vital signs, for example, when the significance level is set to 5%, the test method determines that P<0.05 is “not normally distributed”, and P≥0.05 is “normal distributed”. In addition, the P value is the probability of measuring the evidence for rejecting the null hypothesis.

Using this Shapiro-Wilke test, measurement values that become “outliers” based on P<0.05 are extracted for a set of measurement values of vital signs. That is, this outlier is excluded as a measurement value of a vital sign for which normality is not guaranteed, and only quality data with guaranteed normality is extracted and used for abnormality determination of the vital signs.

In addition, although the cases shown in FIGS. 25 to 28 provide an aspect in which vital signs are measured once a day and vital reference information is set based on vital information for 4 days, for example, an aspect in which vital signs are measured twice a day, once in the morning and once in the afternoon, these are prepared for two days, and the vital reference information is set from the total and four points of vital information may be provided.

In addition, as the measurement values of vital signs, as described above, re-measurement is requested for vital information once determined as “abnormality”, and abnormality determination of vital signs with respect to re-measured vital information, which is the value of vital signs that have been re-measured can also be performed. Thereby, it becomes possible to perform determination using vital information with good accuracy again on the numerical value for which the abnormality determination of vital signs was made due to the poor measurement method or the like. It is also possible to set vital reference information using the re-measurement vital information.

In the cases shown in FIGS. 25 to 28, vital signs were measured once a day, but continuous vital information acquired using, for example, a wearable measuring device that can be worn on the subject's body may also be employed in the acquisition of vital information in the present disclosure.

As described above, the software of the present disclosure reflects vital signs or daily conditions in consideration of the individual differences of the subjects, enables the subjects to determine the intra-individual variation more quickly with high accuracy, and contributes to the subject's health care or the provision of medical care for the characteristics of each individual.

In addition, the health condition determination device of the present disclosure reflects vital signs or daily conditions in consideration of the individual differences of the subjects, enables the subjects to determine the intra-individual variation more quickly with high accuracy, and contributes to the subject's health care or the provision of medicine for the characteristics of each individual.

In addition, the health condition determination method of the present disclosure reflects vital signs or daily conditions in consideration of the individual differences of the subjects, enables the subjects to determine the intra-individual variation more quickly with high accuracy, and contributes to the subject's health care or the provision of medicine for the characteristics of each individual.

### DESCRIPTION OF SYMBOLS

1; Heath condition determination device
1a; Software
2; Calculation unit
2a; Calculation unit
3; Tablet terminal
3a; Input unit (of tablet terminal)
3b; Display screen (of tablet terminal)
3c; Information transmission/reception unit (of tablet terminal)
4; Information recording unit
4a; Information recording unit
5; Reference calculation means
5a; Reference calculation means
6; Determination processing means
6a; Determination processing means
7; Personal information
8; Vital information
9; Reference time information
10; Posture information
11; Temperature information
12; Score determination result information
12a; Vital determination result information
13; Re-measured vital information
14; Mean value calculation means
15; Standard deviation calculation means
16; Normal distribution calculation means
18; Score determination reference information
21a; Vital meter
21b; Thermometer
22a; Smartphone terminal
22b; Personal computer terminal (PC terminal)
23; Information input means
24; Information recording means
24a; Information recording means
30a; Internet
3a; Information management server
32b; Software
32c; Software
32d; Software
50a; User terminal
50b; External terminal
60a; User terminal
60b; External terminal
70b; Management terminal
100; Scoring processing means
100a; Scoring processing means
101; Scoring reference configuring means
102; Scoring reference information
102a; Vital determination reference information
103; Score value information

1. Software for determining health condition of an individual, based on vital information that is a value of a measured vital sign, the software configured to be installed on an information processing device comprising:
an information input means configured to receive an input of vital information, which is measured from the individual and follows a normal distribution, and an input of information on measurement date and time;
an information recording means configured to record the inputted vital information, and the inputted information on measurement date and time;
a reference calculation means configured to calculate a mean μ or a standard deviation σ or both from part or all of pieces of the recorded vital information; and
a determination means configured to determine whether vital information to be determined is an abnormal value, based on a predetermined numerical range which is configured based on the mean μ or the standard deviation σ or both,
wherein the vital information comprises at least one measurement value of body temperature, pulse, blood pressure, or pulse pressure, and
wherein the predetermined numerical range is established for single type of the vital information by using at least four pieces of the vital information for the single type of the vital information recorded in the information recording means, and has a value of Equation (1) below as a lower limit, a value of Equation (2) below as an upper limit, and at least one of the lower limit or the upper limit as a reference, Equation (1) and Equation (2) being expressed using the mean μ, the standard deviation σ, and n and m that are numbers greater than 0 (zero):
μ−nσ Equation (1); and
μ+mσ Equation (2), and
wherein when the predetermined numerical range is established using X pieces of the vital information for single type of the vital information where X is 5 or greater, intra-individual variation of the individual, based on a data group for first four pieces out of the X pieces, is reflected in the predetermined numerical range, and when a test is performed by one-way analysis of variance with respect to the data group for the first four pieces and a data group for the X pieces, no significant difference exists between the data group for the first four pieces and the data group for the X pieces.

2. The software of claim 1, wherein the reference calculation means is configured to calculate the mean μ and the standard deviation σ from the vital information recorded in the information recording means wherein the vital information of the individual is measured twice or more per day for at least 2 days.

3. The software of claim 1, wherein the reference calculation means is configured to calculate the mean μ and the standard deviation σ from the vital information recorded in the information recording means wherein the vital information of the individual is measured for at least 4 days

4. (canceled)

5. The software of claim 1, wherein the predetermined numerical range is configured to comprise the vital information determined as an abnormal value by the determination means.

6. The software of claim 1, wherein the predetermined numerical range is configured to exclude the vital information determined as an abnormal value by the determination means.

7. The software of claim 1, wherein the predetermined numerical range is configured to exclude the vital information to be determined.

8. The software of claim 1, wherein the predetermined numerical range is configured to comprise the vital information to be determined.

9. The software of claim 1, wherein the predetermined numerical range is configured to exclude the vital information measured from a subject in a predetermined state.

10. The software of claim 1, wherein the information input means is configured to receive an input of re-measured vital information of the individual, and an input of a measurement date and time after the determination means determines that the vital information to be determined is an abnormal value, and
wherein the determination means is configured to determine whether the re-measured vital information is an abnormal value.

11. A health condition determination device for determining a health condition of an individual, based on vital information that is a value of a measured vital sign, the health condition determination device comprising:
an information input means configured to receive an input of vital information, which is measured from the individual and follows a normal distribution and an input of information on measurement date and time;
an information recording means configured to record the inputted vital information and the inputted information on measurement date and time;
a reference calculation means configured to calculate a mean μ or a standard deviation σ or both from part or all of pieces of the recorded vital information;
a determination means configured to determine whether the vital information to be determined is an abnormal value, based on a predetermined numerical range which is configured based on the mean μ or the standard deviation σ or both; and
a display means capable of displaying a determination result determined by the determination means,
wherein the vital information comprises at least one measurement of body temperature, pulse, blood pressure, or pulse pressure, and
wherein the predetermined numerical range is established for single type of the vital information by using at least four pieces of the vital information for in the single type of the vital information recorded in the information recording means and has a value of Equation (1) below as a lower limit, a value of Equation (2) below as an upper limit, and at least one of the lower limit or the upper limit as a reference, Equation (1) and Equation (2) being expressed using the mean μ, the standard deviation σ, and n and m that are numbers greater than 0 (zero):
μ−nσ Equation (1); and
μ+mσ Equation (2), and
wherein when the predetermined numerical range is established using X pieces of the vital information for single type of the vital information where X is 5 or greater, intra-individual variation of the individual, based on a data group for first four pieces out of the X pieces, is reflected in the predetermined numerical range, and when a test is performed by one-way analysis of variance with respect to the data group for the first four pieces and a data group for the X pieces, no significant difference exists between the data group for the first four pieces and the data group for the X pieces.

12. A health condition determination method which is a method executed by a computer and determines a health condition of an individual, based on vital information which is measured values of vital signs, the health condition determination method comprising:
a reference calculating step of calculating at least a mean μ or a standard deviation σ or both from a predetermined number of vital information or more out of vital information which is measured from the individual and follows a normal distribution; and
a determination step of determining whether vital information to be determined is an abnormal value, based on a predetermined numerical range which is configured based on the mean μ or the standard deviation σ or both,
wherein the vital information comprises at least one measurement value of body temperature, pulse, blood pressure, or pulse pressure, and
wherein the predetermined numerical range is established for each single type of vital information by using at least four pieces of the vital information for each single type of vital information recorded in the information recording means, and has a value of Equation (1) below as a lower limit, a value of Equation (2) below as an upper limit, and at least one of the lower limit or the upper limit as a reference, Equation (1) and Equation (2) being expressed using the mean μ, the standard deviation σ, and n and m that are numbers greater than 0 (zero):
μ−nσ Equation (1); and
μ+mσ Equation (2), and
wherein when the predetermined numerical range is established using X pieces of the vital information for single type of the vital information where X is 5 or greater, intra-individual variation of the individual, based on a data group for first four pieces out of the X pieces, is reflected in the predetermined numerical range, and when a test is performed by one-way analysis of variance with respect to the data group for the first four pieces and a data group for the X pieces, no significant difference exists between the data group for the first four pieces and the data group for the X pieces.

13. Software for scoring vital information that is a value of an obtained vital sign and determining the health condition of an individual, based on the obtained score result information, the software configured to be installed on an information processing device comprising:
an information input means configured to receive an input of vital information, which is acquired from the individual and follows a normal distribution and an input of information on acquisition date and time;
an information recording means configured to record the inputted vital information and the inputted information on acquisition date and time;
a reference calculation means configured to calculate a mean μ or a standard deviation σ or both from part or all of pieces of the recorded vital information;
a scoring processing means configured to score input predetermined vital information to calculate score result information which is a value of a score, based on a predetermined scoring condition; and
a score determination means configured to determine whether the score result information is an abnormal value, based on a predetermined score determination condition,
wherein the vital information comprises at least one measurement value of body temperature, pulse, blood pressure, or pulse pressure, and
wherein with respect to the at least one measurement value of body temperature, pulse, blood pressure, or pulse pressure, the predetermined scoring condition is established for each single type of vital information by using at least four pieces of the vital information for the single type of the vital information, and has a value of Equation (1) below as a lower limit, a value of Equation (2) below as an upper limit, and at least one of the lower limit or the upper limit as a reference, Equation (1) and Equation (2) being expressed using the mean μ, the standard deviation σ, and n and m that are numbers greater than 0 (zero):
μ−nσ Equation (1); and
μ+mσ Equation (2), and
wherein when the predetermined scoring condition is established using X pieces of the vital information for single type of the vital information where X is 5 or greater, intra-individual variation of the individual, based on a data group for first four pieces out of the X pieces, is reflected in the predetermined scoring condition, and when a test is performed by one-way analysis of variance with respect to the data group for the first four pieces and a data group for the X pieces, no significant difference exists between the data group for the first four pieces and the data group for the X pieces.

14. The software of claim 13, wherein the reference calculation means is configured to calculate the mean μ and the standard deviation σ from the vital information recorded in the information recording means wherein the vital information of the individual is measured twice or more per day for at least 2 days.

15. The software of claim 13, wherein the reference calculation means is configured to calculate the mean μ and the standard deviation σ from the vital information recorded in the information recording means wherein the vital information of the individual is measured for at least 4 days.

16. The software of claim 13, wherein the vital information comprises:
at least one measurement value of body temperature, pulse, pulse pressure, or respiratory rate;
a measurement value of oxygen saturation; and
a consciousness level evaluation result obtained by observing a consciousness level,
wherein the scoring condition is a predetermined numerical range set in advance with respect to the measured value of oxygen saturation, and
wherein the scoring condition is a predetermined observation state indicating a degree of consciousness level with respect to the consciousness level evaluation result.

17. The software of claim 13, wherein the score determination means is configured to determine whether it is an abnormal value, at least, with respect to the total points of the score result information obtained by scoring multiple types of vital information.

18. The software of claim 13, wherein the predetermined scoring condition is configured to comprise the vital information which is a calculation basis of the score result information determined as an abnormal value by the score determination means.

19. The software of claim 13, wherein the predetermined scoring condition is configured to exclude the vital information which is a calculation basis of the score result information determined as an abnormal value by the score determination means.

20. The software of claim 13, wherein the predetermined scoring condition is configured to exclude vital information to be determined.

21. The software of claim 13, wherein the predetermined scoring condition is configured to comprise vital information to be determined.

22. The software of claim 13, wherein the predetermined scoring condition is configured to exclude the vital information measured from a subject under a predetermined state.

23. A health condition determination device for scoring vital information that is a value of an obtained vital sign and determining the health condition of an individual, based on the obtained score result information, the health condition determination device comprising:
an information input means configured to receive an input of vital information acquired from the individual and follows a normal distribution and an input of information on acquisition date and time;
an information recording means configured to record the inputted vital information and the inputted information on acquisition date and time;
a reference calculation means configured to calculate a mean μ or a standard deviation σ or both from part or all of pieces of recorded vital information;
a scoring processing means configured to score input predetermined vital information to calculate score result information which is a value of a score, based on a predetermined scoring condition;
a score determination means configured to determine whether the score result information is an abnormal value, based on a predetermined score determination condition; and
a display means capable of displaying a determination result determined by the score determination means,
wherein the vital information comprises at least one measurement value of body temperature, pulse, blood pressure, or pulse pressure, and wherein with respect to the at least one measurement value scicctcd fr mof body temperature, pulse, blood pressure, or pulse pressure, the predetermined scoring condition is established for each single type of vital information by using at least four pieces of the vital information for the single type of the vital information, and has a value of Equation (1) below as a lower limit, a value of Equation (2) below as an upper limit, and at least one of the lower limit or the upper limit as a reference, Equation (1) and Equation (2) being expressed using the mean μ, the standard deviation σ, and n and m that are numbers greater than 0 (zero):
μ−nσ Equation (1); and
μ+mσ Equation (2), and
wherein when the predetermined scoring condition is established using X pieces of the vital information for single type of the vital information where X is 5 or greater, intra-individual variation of the individual, based on a data group for first four pieces out of the X pieces, is reflected in the predetermined scoring condition, and when a test is performed by one-way analysis of variance with respect to the data group for the first four pieces and a data group for the X pieces, no significant difference exists between the data group for the first four pieces and the data group for the X pieces.

24. A health condition determination method, which is a method executed by a computer, scores vital information that is a value of an obtained vital sign, and determines the health condition of an individual, based on the obtained score result information, the health condition determination method comprising:
an information recording step of receiving and recording an input of vital information obtained from an identical individual and following a normal distribution;
a reference calculating step of calculating a mean p and a standard deviation σ of all or a part of the multiple recorded vital information;
a scoring processing step of scoring the input predetermined vital information to calculate score result information which is a value of a score, based on a predetermined scoring condition; and
a score determining step of determining whether the score result information is an abnormal value, based on the predetermined score determination condition,
wherein the vital information comprises at least one measurement value of body temperature, pulse, blood pressure, or pulse pressure, and
wherein with respect to the at least one measurement value of body temperature, pulse, blood pressure, or pulse pressure, the predetermined scoring condition is established for each single type of vital information by using at least four pieces of the vital information for the single type of vital information, and has a value of Equation (1) below as a lower limit, a value of Equation (2) below as an upper limit, and at least one of the lower limit or the upper limit as a reference, Equation (1) and Equation (2) being expressed using the mean μ, the standard deviation σ, and n and m that are numbers greater than 0 (zero):
μ−nσ Equation (1); and
μ+mσ Equation (2), and
wherein when the predetermined scoring condition is established using X pieces of the vital information where X is 5 or greater, intra-individual variation of the individual, based on a data group for first four pieces out of the X pieces, is reflected in the predetermined scoring condition, and when a test is performed by one-way analysis of variance with respect to the data group for the first four pieces and a data group for the X pieces, no significant difference exists between the data group for the first four pieces and the data group for the X pieces.
